(19) United States
(12) Patent Application Publication
(10) Pub. No.: US 2024/0277737 A1
(43) Pub. Date: Aug. 22, 2024

(54) METHOD FOR GENERATING DRY POWDER SUITABLE FOR INHALATION ADMINISTRATION OF ANTIMICROBIAL PHOSPHOLIPID COMPOSITIONS

(71) Applicant: Celestial Therapeutics, Inc., Irvine, CA (US)

(72) Inventors: Ajay Gupta, Irvine, CA (US); Luca Serratore, Barcellona (ES); Jack Sorrell, Nottingham (GB); Jasmine Ahad, Langworth (GB)

(21) Appl. No.: 18/686,622

(22) PCT Filed: Aug. 26, 2022
(86) PCT No.: PCT/US22/41764

§ 371 Date: Feb. 26, 2024

Related U.S. Application Data
(60) Provisional application No. 63/237,283, filed on Aug. 26, 2021.

(51) Int. Cl. A61K 31/683; A61K 9/00; A61K 9/16

(52) U.S. Cl. CPC A61K 31/683; A61K 9/0075; A61K 9/1688

(57) ABSTRACT

A method for treating an infection in a subject is provided. The method includes administering to the subject, via an oral inhalation route, an amount of a composition that is effective to inhibit the infection. The composition contains a phosphatidylglycerol such as POPG.

### CROSS-REFERENCE TO RELATED APPLICATION

This application is a national stage filing of PCT/US22/41764, filed on Aug. 26, 2022, having the same inventors and the same title, and which is incorporated herein by referenced in its entirety; which application claims the benefit of priority from U.S. Provisional Application No. 63/232,423, filed Aug. 26, 2021, having the same inventor and the same title, and which is incorporated herein by reference in its entirety.

### FIELD OF THE DISCLOSURE

The present disclosure relates generally to antimicrobial phospholipid compositions, and more particularly to methods for generating and administering the same via inhalation.

### BACKGROUND OF THE DISCLOSURE

POPG is a pulmonary surfactant phospholipid that reduces interfacial tension at the air/water interfaces in the alveoli. This prevents the tension from pulling the alveolar membranes in-wards which would collapse them and lead to respiratory distress. (Pubchem, 2021).

In a recent study it was determined that POPG, could markedly attenuate inflammatory responses induced by lipopolysaccharide through direct interactions with the Toll-like receptor 4 (TLR4) interacting proteins CD14 and MD-2. CD14 and TLR4 have been implicated in the host response to Respiratory Syncytial Virus (RSV). Treatment of bronchial epithelial cells with POPG significantly inhibited interleukin-6 and -8 production, as well as the cytopathic effects induced by RSV (Numata, et al., 2009).

### SUMMARY OF THE DISCLOSURE

In one aspect, method for treating an infection or inflammation in a subject is provided. The method comprises administering to the subject, via an oral inhalation route, an amount of a composition that is effective to inhibit said infection or said inflammation, wherein the composition contains a phosphatidylglycerol.

### BRIEF DESCRIPTION OF THE DRAWINGS

FIG. 1 is an illustration of the skeletal formula of 1-Palmitoy-2-loleoyl-sn-glycerol-3-phospho-(1′-rac-glycerol) (POPG).

FIG. 2 is a graph of distribution coefficient (log D) vs pH for POPG.

FIG. 3 is a graph of dissociation constant (pKa) vs pH for POPG.

FIG. 4 is an isoelectric point chart (charge as a function of pH) for POPG.

FIG. 5 is the pKa structure of POPG.

FIG. 6 is a graph of the aqueous solubility (in mg/ml) of POPG as a function of pH.

FIG. 7 is a cubic model of graph of peak response (in mAU) as a function of concentration (in μg/mL).

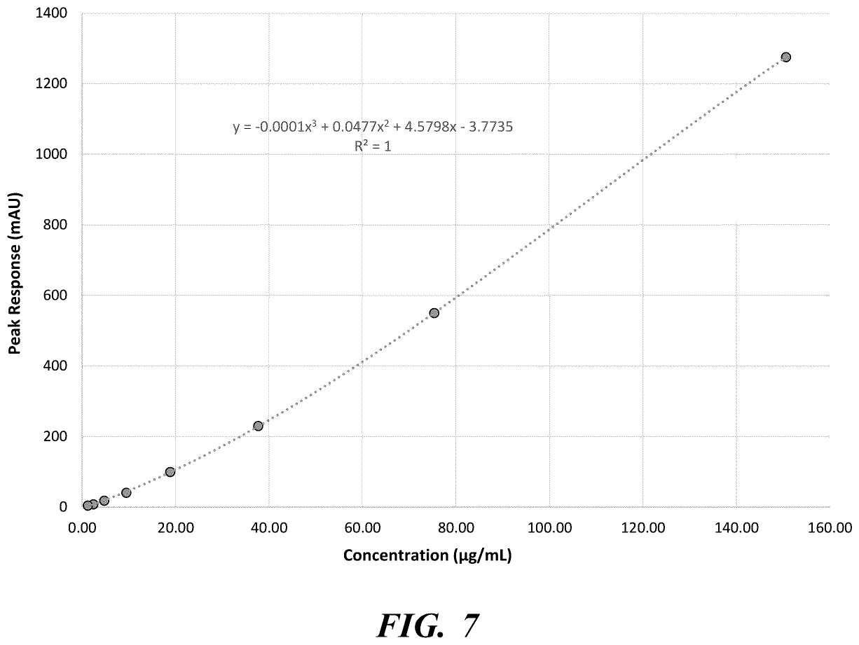

FIG. 8 is a chromatogram of the Limit of Quantification (LOQ).

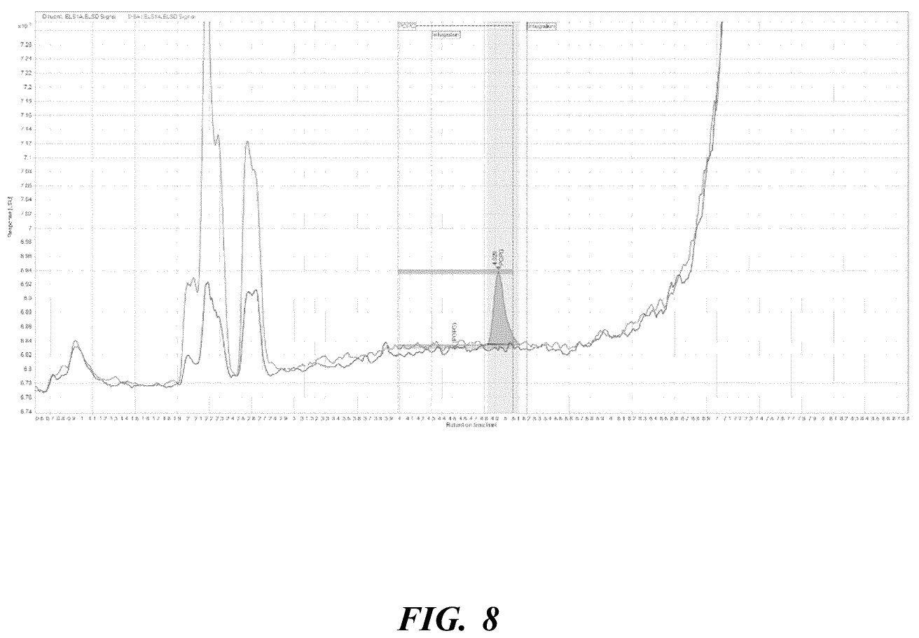

FIG. 9 is a chromatogram of the Limit of Detection (LOD).

FIG. 10 is chromatogram of palmitic and oleic acid.

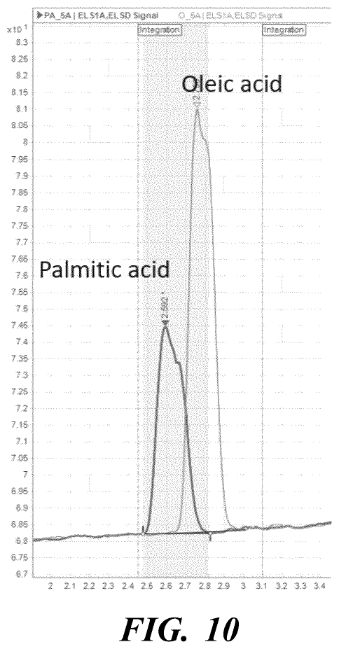

FIG. 11 is chromatogram of POPG and phosphatidic acid.

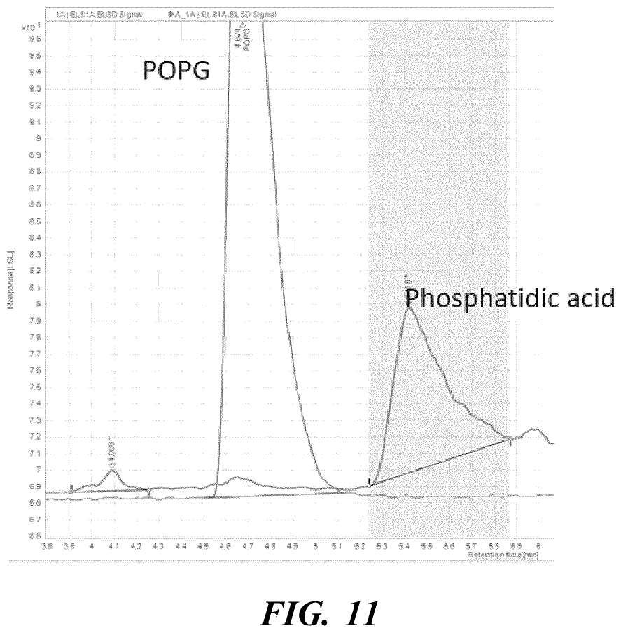

FIG. 12 is an exemplary chromatogram of the plate stage 7 for coating agent specificity.

FIG. 13 is a plot of mass deposition as a function of stage for the aerodynamic particle size distribution (APSD) deposition of POPG with and without a coating agent (the coating agent was Tween 20 in ethanol).

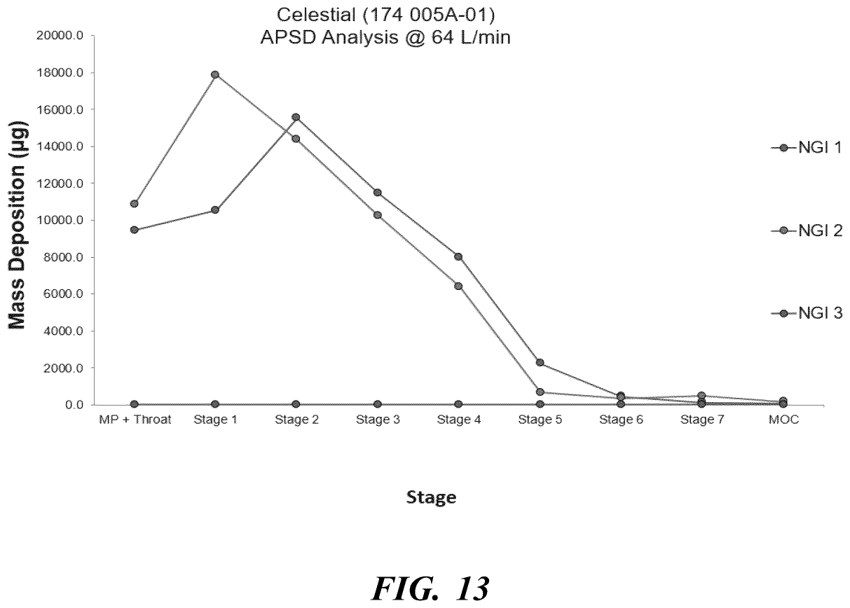

FIG. 14 is a graph of particle size distribution of POPG raw material.

FIG. 15 is a graph of the Differential Scanning calorimetry (DSC) results of POPG raw material.

FIG. 16 is the X-ray powder diffraction (XRPD) pattern of POPG raw material.

FIG. 17 is a series of scanning electron micrographs (SEMs) of the POPG raw material shown at magnifications of 100 μm (FIG. 17a), 10 μm (FIG. 17b), 5 μm (FIG. 17c) and 1 μm (FIG. 17d).

FIG. 18 is a graph of the particle size distribution (PSD) of a first batch of a spray drying product containing POPG.

FIG. 19 is a graph of the PSD of a batch of a spray drying product containing POPG.

FIG. 20 is a graph of the PSD of a batch of a spray drying product containing POPG.

FIG. 21 is a graph of the PSD of a batch of spray drying product containing POPG; FIG. 21a shows the initial PSD, and FIG. 21b shows the PSD following storage at −20° C.

FIG. 22 is a graph of the PSD of a batch of spray drying product containing POPG; FIG. 22a shows the initial PSD, and FIG. 22b shows the PSD following storage at −20° C.

FIG. 23 is a graph of the PSD of a batch of spray drying product containing POPG; FIG. 23a shows the initial PSD, and FIG. 23b shows the PSD following storage at −20° C.

FIG. 24 is a series of SEMs of a spray dried product containing POPG shown at magnifications of 100 μm (FIG. 24a), 10 μm (FIG. 24b), 5 μm (FIG. 24c) and 1 μm (FIG. 24d).

FIG. 25 is a series of SEMs of a spray dried product containing POPG shown at magnifications of 100 μm (FIG. 25a), 10 μm (FIG. 25b), 5 μm (FIG. 25c) and 1 μm (FIG. 25d).

FIG. 26 is a series of SEMs of a spray dried product containing POPG shown at magnifications of 100 μm (FIG. 26a), 10 μm (FIG. 26b), 5 μm (FIG. 26c) and 1 μm (FIG. 26d).

FIG. 27 is an XPRD a spray dried product containing POPG and that of POPG raw material.

FIG. 28 is an XPRD a spray dried product containing POPG and that of POPG raw material.

FIG. 29 is an XPRD a spray dried product containing POPG and that of POPG raw material.

FIG. 30 is a DSC of a spray dried product containing POPG post spray drying.

FIG. 31 is a DSC of a spray dried product containing POPG post spray drying.

FIG. 32 is a DSC of a spray dried product containing POPG post spray drying.

FIG. 33 is a DSC of a spray dried product containing POPG post spray drying.

FIG. 34 is a TAM analysis profile of a spray dried product containing POPG post spray drying.

### DETAILED DESCRIPTION

It has now been found that the foregoing needs may be met with an inhaled product containing 1-Palmitoy-2-loleoyl-sn-glycerol-3-phospho-(1′-rac-glycerol) (POPG)-Surfactant. This product may be utilized for various pulmonary applications such as, for example, Respiratory Syncytial virus, H1N1, human rhinovirus infection treatment and prophylaxis as well as for treatment of Allergic Asthma, COPD, Sepsis, ALI, CF etc.

In order to develop the foregoing product, a Technical Product Profile (TPP) was established for the product, and a chemical-informatics analysis was conducted to understand the potential formulation routes that could be taken to establish a product that matches the desired TPP. Solubility for POPG was found to be at a minimum level between approximately pH 0.0 and pH 12.0. The analysis showed that the aqueous solubility of POPG increases significantly in alkaline solutions and reached the maximum solubility (in excess of ˜580 mg·mL) at a pH of 14.0. This appears to be due to the deprotonation of basic groups at high pH, resulting in a negative charge, therefore increasing the aqueous. Based on the lipid nature of the API and the chemical-informatics results, the usage of an organic solvent to solubilize the POPG as methanol, ethanol, dichloromethane, chloroform was suggested.

An Assay method for POPG was then successfully developed to quantify the emitted dose. The calibration curve in the range between 1.14-146.29 μg/mL, as a free base, assessed follows a cubic regression model. The signal to noise (SN) for Limit of quantification (LOQ) and Limit of detection (LOD) were 16.0 and 7.5 at the concentrations 2.35 μg/mL and 1.14 μg/mL, respectively. Also, no peak in the diluent interferes with the API peak. The HPLC method passed all the other system suitability requirements: specificity, precision, standard agreement.

The POPG raw material was then characterized through Differential Scanning calorimetry (DSC), Scanning Electron Microscopes (SEM) and Particle Size Distribution (PSD). Based on the POPG characterization, three spray dried formulations were manufactured adjusting the process parameters to obtain a PSD between 2-5 μm. The residual solvent for Dichloromethane (DCM) and Methanol (MeOH) was lower than the LOQ. The batch 174 #005A-01 manufacture showed the best properties in terms of PSD, with a Dv50 of 2.46 μm. The Spray drying process as well did not seem to have an impact on the degree of crystallinity of the material. The SEM analysis showed that this powder had smaller aggregates compared with the other two batches manufactured and presented spherical particles of different particle size. It was observed that the storage conditions and the re-equilibration time of the powder are key factors that impacts the PSD.

Based on this previous observation, a new batch was manufactured using the same spray drying parameters used for the product 174#005A-01. The PSD was evaluated through time and after 14 days storage and re-equilibration it was observed no variation on the PSD results when compared to the time zero analysis. The DSC analysis did not show any transitions phase comparing the powder spray drying and after 14 days storage.

A single 7 g batch of POPG was successfully micronized and the PSD evaluated.

An assay evaluation was performed on 174 #010A-01 and 174 #013A-01 and no difference was observed. Although there was no difference on the assay data, the 174 #013A-01 showed different results on the PSD when evaluate through time.

The bulk density of the 174 #010A-01 and 174 #013A-01 showed as well similar bulk density based on the capsule filling obtained.

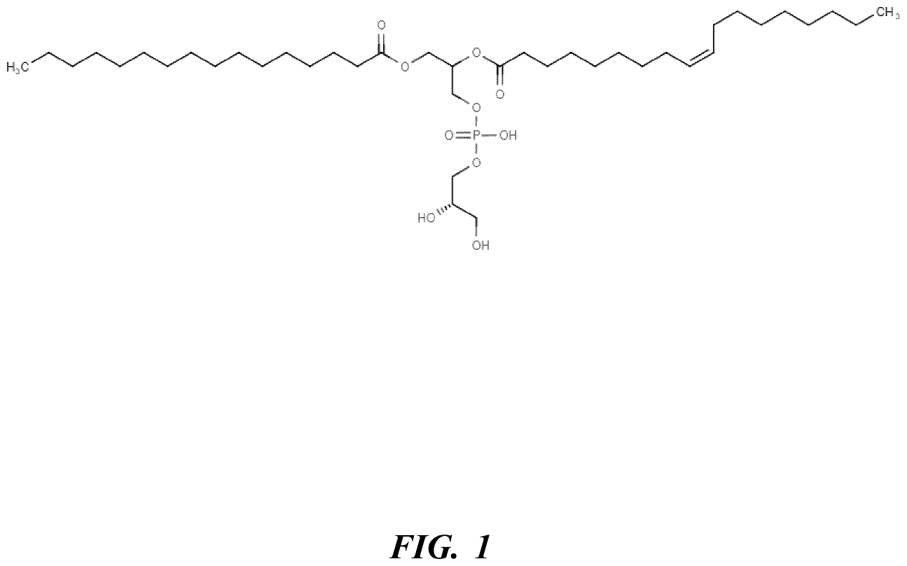

As previously noted, POPG (the skeletal formula of which is depicted in FIG. 1) is a pulmonary surfactant phospholipid that reduces interfacial tension at the air/water interfaces in the alveoli. This prevents the tension from pulling the alveolar membranes in-wards which would collapse them and lead to respiratory distress. (Pubchem, 2021). In a recent study it was determined that POPG, could markedly attenuate inflammatory responses induced by lipopolysaccharide through direct interactions with the Toll-like receptor 4 (TLR4) interacting proteins CD14 and MD-2. CD14 and TLR4 have been implicated in the host response to Respiratory Syncytial Virus (RSV). Treatment of bronchial epithelial cells with POPG significantly inhibited interleukin-6 and -8 production, as well as the cytopathic effects induced by RSV (Numata, et al., 2009).

This product was explored for orally inhaled delivery as a powder. The usage of powder medication formulations can offer advantages, including greater stability than liquid formulations and potential that preservatives may not be required. A number of factors such as moisture sensitivity, solubility, particle size, particle shape, and flow characteristics will impact deposition and absorption (Djupesland Per Gisle, 2013).

About 80 mg of drug substance powder was hand filled into a DPI system for a target delivered dose between 50 mg. The weight of the system was taken before and after filling to confirm accurate fill weight of 80 mg+/−0.2 mg. Powder understanding from the feasibility studies was utilized to help define the filling and storage environmental conditions. A summary of the quality target product profile can be found in TABLE 1.

TABLE 1
Quality target product profile (QTPP).

| Quality Attribute | Target |
|---|---|
| Intended clinical use | Treatment or prophylaxis of RSV, H1N1 and HRV infections. |
| Route of administration | Inhalation route. |
| Dosage form | Dry powder inhaler (multidose) |
| Dosage strength | 80 mg |
| Emitted dose | 50 mg |
| Formulation | Spray dried formulation. |
| Drug product quality attributes | DPI: Assay, impurities, residual solvents, DDU, APSD, |
| | DSD and water content to be monitored. |
| Container closure system | DPI device in aluminium foil pouches with desiccant |

The samples outlined in TABLE 2 were used during this study.

TABLE 2
Outline of material to be used.

| Material | Batch No. | Quantity |
|---|---|---|
| 1-palmitoyl-2-oleoyl-sn-glycero-3- | TBD | 200 g |
| phospho-(1′rac-glycerol) (sodium salt) | | |
| Ethanol | TBD | 5 L |
| Dichloromethane | TBD | 5 L |
| Ethyl-acetate | TBD | 5 L |
| DPI device | TBD | 100 |
| Methanol | TBD | 5 L |
| Isopropanol | TBD | 5 L |

### Methods

### Delivered Dose Uniformity (DDU) Analysis

A Delivered dose uniformity was developed as stipulated for DPI products in the USP <601> and FDA guidance (FDA, 2002) (USP-NF, 2014).

### Aerodynamic Particle Size Analysis (APSD)

The Next Generation Impactor (NGI) with USP throat was developed to determine the APSD for the DPI device performed as stipulated for DPI products in the USP <601> and in the FDA guidance (USP-NF, 2014) (FDA, 2018)

### Assay by UPLC-ELSD

Assay method was assessed with UPLC coupled with and ELSD detector. This method was used as a fast quantitative method for APSD and DDU.

Differential Scanning calorimetry (DSC)

DSC analysis was performed using a TA Instruments Q20 MDSC with auto sampler and refrigerated cooling accessory. Approximately 1-7 mg of sample were tested in an aluminium pan under an N₂ flow (50 mL/min). Pans were sealed using a T Zero pan press. The following cycle was used:
Data storage: Off
Equilibrate at −20.00° C.
Isothermal for 5.00 min
Data storage: On
Ramp 10.00° C./min to 250.00° C.
Isothermal for 5.00 min
Ramp 10.00° C./min to −20.00° C.
Isothermal for 5.00 min
Ramp 10.00° C./min to 250.00° C.
End of method
   Data analysis was undertaken using TA instrument Universal Analysis 2000 software (build 4.5.0.5). The calorimeter head was continuously flushed with dry nitrogen gas at 0.2 L/min during all measurements.

### Scanning Electron Microscopy (SEM)

The particle size and surface morphology of POPG was studied by SEM. Samples were analyzed using a JEOL 6490LV SEM and images taken at a range of magnifications (×200, ×1000, ×5000, and ×10000).

Sample powders were scattered onto a sticky carbon tab on a SEM stub. Excess was removed using a compressed air duster and the sample stubs were then coated in Platinum with a Polaron SC 7640 sputter coater for 90 s at 2.2 kv, 15 mA (approximately 12-15 nm) Samples were then transferred to the SEM for image analysis where they were imaged in high vacuum (HV mode).

### X-ray Powder Diffraction (XRPD)

To determine the X-ray powder diffraction (XRPD) pattern of the POPG samples, all samples were analysed using a Malvern PANlytical Xpert MPD operating in Bragg Brentano geometry equipped with PIXcel area detector and a sealed tube Cu source operating at 40 kV and 40 mA. A Johansson focussing beam monochromator selected pure Kα1 radiation which was then passed through 0.04 rad Soller slits to reduce axial beam divergence Programmable Divergence Slits and Programmable Anti Scatter Slits were used in automatic mode to match the prepared sample length A fixed mask matching the prepared sample width and a fixed 1 anti scatter slit were used in the incident beam path.

### Water Content

The water contend determination was carried out using a Karl-Fischer (KF) titration. The powder was placed inside an oven which will transfer the water into the titration vessel. Common KF solvents were used.

### Residual Solvent Analysis by Gas Chromatography

The residual solvent of the spray dried formulations was carried out using a headspace gas chromatography system.

The basic properties were calculated and presented in Table 3 below.

TABLE 3
Basic Properties of POPG

| Property | Value |
|---|---|
| IUPAC Name | [(2R)-1-[2,3-dihydroxypropoxy(hydroxy)phosphoryl]oxy- |
| | 3-hexadecanoyloxypropan-2-yl] (Z)-octadec-9-enoate |
| Molar Mass | 749.02 g/mol |
| Exact Mass | 748.5254 Da |
| Formula | C40H77O10P |
| Composition | C (64.14%), H (10.36%), O (21.36%), P (4.14%) |
| Lipinski' Rule 5 | FALSE |

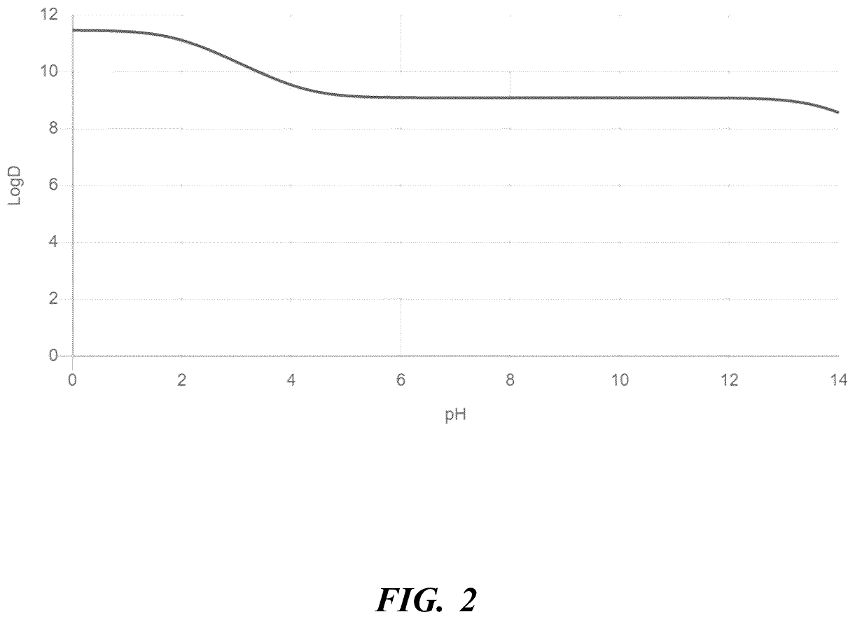

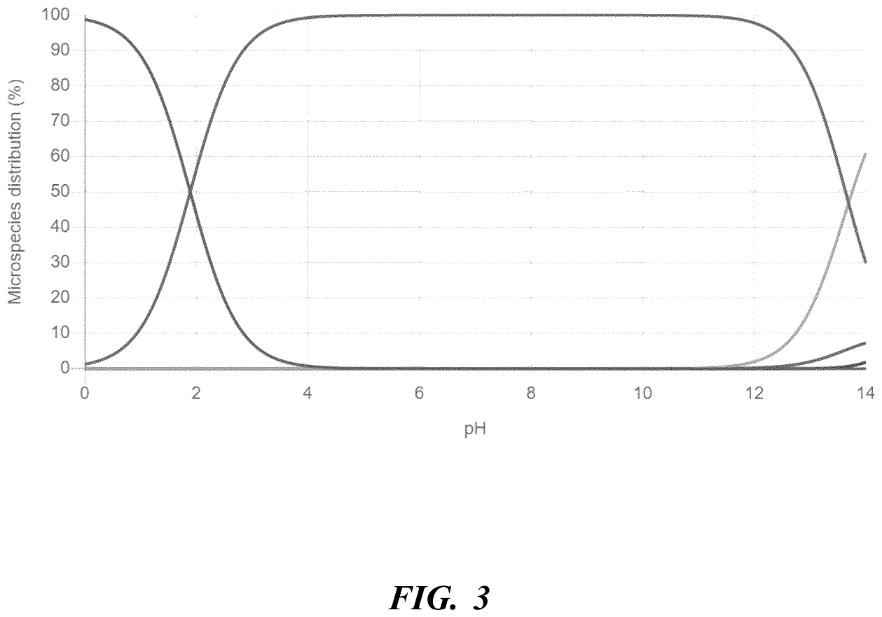

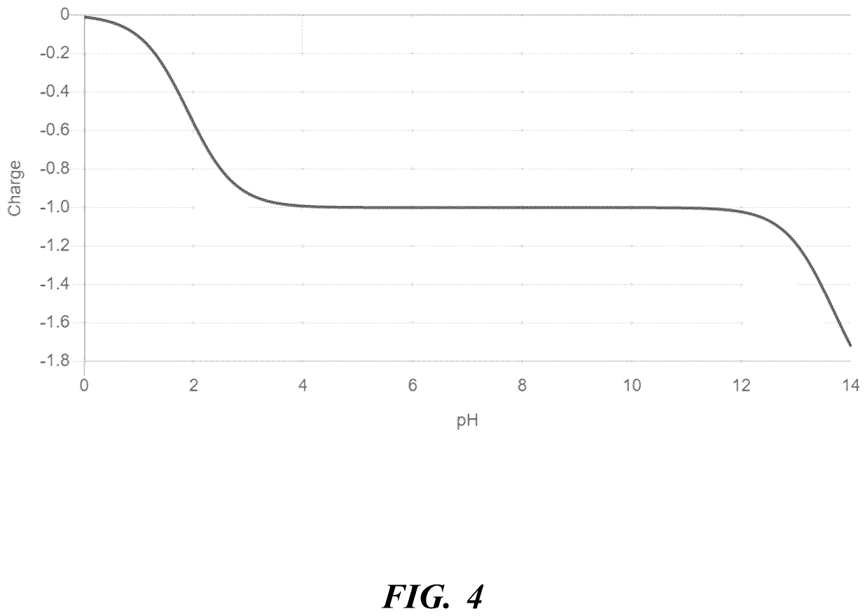

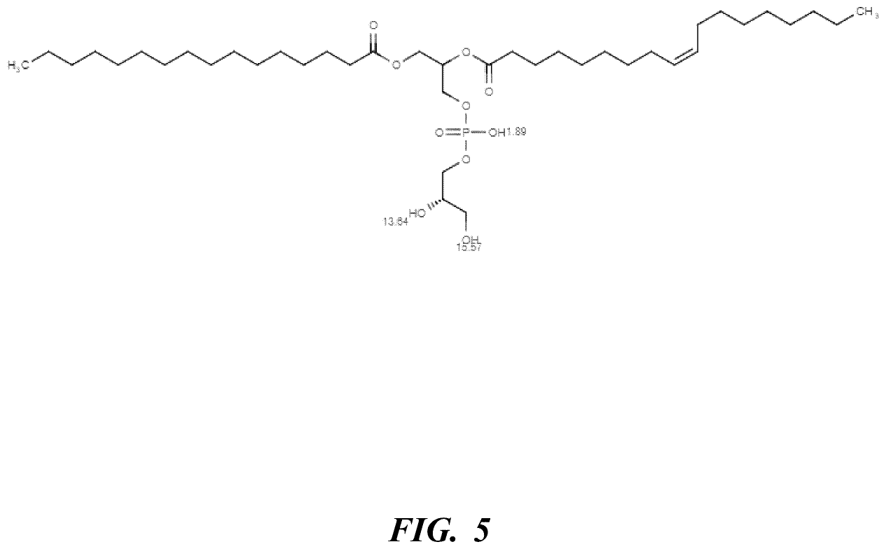

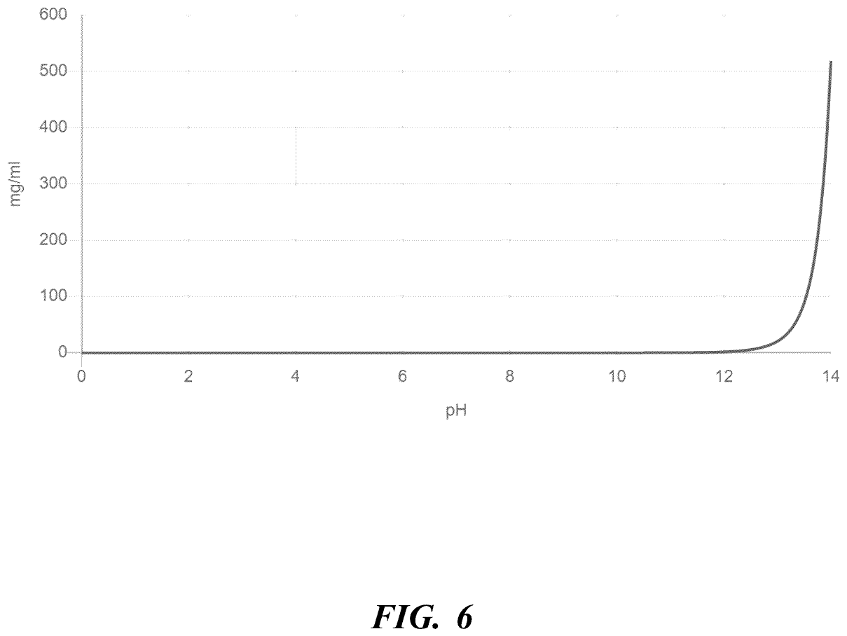

Data regarding how the distribution coefficient (log D), dissociation constant (pKa), and aqueous solubility of POPG vary across the pH range are given in FIGS. 2-6.

The analysis shows that the aqueous solubility of POPG increases significantly in alkaline solutions where the pH is 14.0 with maximum aqueous solubility (in excess of ˜580 mg·mL). Solubility is at a minimum level between approximately pH 0.0 and pH 12.0. This appears to be due to the deprotonation of basic groups at high pH, resulting in a negative charge, therefore increasing the aqueous. Based on the nature of the lipidic nature of the API and the chemical-informatic analysis results, the usage of an organic solvent to solubilize the POPG as methanol, ethanol, dichloromethane, chloro-form was suggested.

### HPLC Assay/RS Method Assessment

A UPLC/ELSD method was evaluated for the quantification of the POPG. The details of the preparation of the solution is summarised in the document under the ID PP-185-502-POPGnasal v 2.0.

### POPG Stock Solution (150 Ug/mL, 145.73 Ug/mL as a Free Base)

A POPG stock solution was prepared with the following characteristics:
POPG sodium salt molecular weight=770.99 g/mol
POPG free base molecular weight=749.02 g/mol
Base/salt ratio=0.972.

### Preparation of the Stock Standard Solutions

Two 30.00 mg+0.5 mg samples of POPG standard were accurately weighed, in duplicate, into two separate 200 mL volumetric flasks. About 40 mL of diluent was added and the resulting solution was sonicated until full dissolution was achieved. The solution was allowed to cool down and make to volume with diluent. The flask was shaken well to homogenise the solution. These are the POPG standard solution A and B at a nominal concentration of 500 μg/mL 150 μg/mL, 145.73 μg/mL as a free base).

### Linearity, Standard Agreement and System Precision

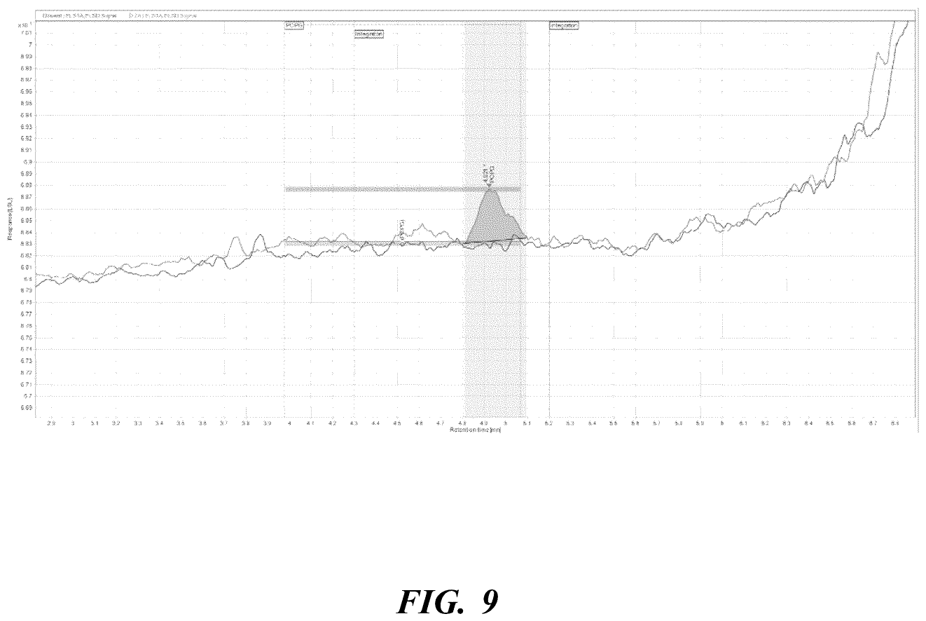

In order to create a range of standards to form a calibration curve, a serial dilution of the stock solutions was prepared as per TABLE 5. The calibration curve results are summarised in TABLES 6-8 and FIG. 9.

The linearity results for this method transfer are presented on TABLE 6. The calibration curve in the range between 1.18-150.58 μg/mL, as a free base, was assessed follows a cubic regression model. The optimal model was selected under the values of the R2 (determination coefficient) The SN for LOQ and LOD were evaluated based on the peak height of the POPG. The LOQ and LOD were 16 and 7.5 at the concentrations 2.35 μg/mL and 1.18 μg/mL, respectively. In addition, no peak in the diluent (or from the coating agents used to perform the APSD) interferes with the API peak. Moreover, the two standards are in agreement and present repeatability (see TABLE 7). The UPLC-ELSD method permitted to distinguish the main analyte from the degradation product.

An assay evaluation was performed on the POPG raw material and is summarised in Table 4. The purity will be evaluated as well with the LC-MS.

TABLE 4
Assay evaluation.

| Material ID | Assay (%) |
|---|---|
| POPG raw material | 101.82 ± 0.68 |

TABLE 5
Working standards calibration curve.

| | Solution to be | Standard volume | Final volume | Resulting conc. (μg/mL) |
|---|---|---|---|---|
| Standard ID | diluted | (mL) | (mL) | POPG sodium salt |
| Stock solution | — | — | — | 150.58 |
| Standard 1A | Stock A | 5 | 10 | 75.29 |
| Standard 2A | Standard 1A | 5 | 10 | 37.64 |
| Standard 3A | Standard 2A | 5 | 10 | 18.82 |
| Standard 4A | Standard 3A | 5 | 10 | 9.41 |
| Standard 5A | Standard 4A | 5 | 10 | 4.71 |
| Standard 6A | Standard 5A | 5 | 10 | 2.35 |
| Standard 7A | Standard 6A | 5 | 10 | 1.18 |

Standard 1B

| | | | | |
|---|---|---|---|---|
| Stock solution | — | — | — | 150.58 |
| B | | | | |

TABLE 6
Calibration curve results

| Concentration | | | | | |
|---|---|---|---|---|---|
| (μg/mL) | Area inj. 1 | Area inj. 2 | Area inj. 3 | Average | % RSD |
| 150.58 | 1264.31 | 1277.02 | 1283.48 | 1274.94 | 0.76 |
| 75.29 | 547.13 | 548.94 | 552.87 | 549.65 | 0.53 |
| 37.64 | 231.55 | 227.35 | 229.80 | 229.57 | 0.92 |
| 18.82 | 99.02 | 98.44 | 99.84 | 99.10 | 0.71 |
| 9.41 | 40.99 | 39.85 | 40.94 | 40.59 | 1.59 |
| 4.71 | 18.32 | 17.15 | 18.43 | 17.97 | 3.94 |
| 2.35 | 8.08 | 6.93 | 7.41 | 7.47 | 7.70 |
| 1.18 | 3.68 | 4.11 | 3.61 | 3.80 | 7.10 |

| | |
|---|---|
| Correlation | 1.00 |

TABLE 7
Standard agreement results.

| Linearity Solution (μg/mL) | Replicate | Area inj. | Average | % RSD |
|---|---|---|---|---|
| WSA | 1 | 1310.61 | 1308.76 | 1.59 |
| | 2 | 1322.23 | | |
| | 3 | 1341.00 | | |
| | 4 | 1283.43 | | |
| | 5 | 1303.34 | | |
| | 6 | 1291.98 | | |
| WSB | 1 | 1282.99 | 1299.65 | 1.81 |
| | 2 | 1316.30 | | |
| Standard Agreement | 100.3 | | | |

TABLE 8
System Suitability.

| Parameter | Acceptance Criteria | Results |
|---|---|---|
| Limit of | TheS/N ratio for the LOQ should | 16 (at the concentration 2.35 μg/mL) |
| Quantification | be NLT 10. | |
| (LOQ) | | |
| Limit of Detection | S/N ratio of the active at a known | 7 (at the concentration 1.18 μg/mL) |
| (LOD) | low should be NLT 3. | |
| Linearity | The correlation co-efficient should | 1.00 between |
| | ideally be NLT 0.99. | 1.18 μg/mL-150.58 μg/mL |
| Standard | Between 98.0-102.0% | 100.3 |
| Verification | | |
| Working precision | % RSD NMT 2.0% | 1.59 |
| (Initial 6 WSA | | |
| injections) | | |
| Specificity | PASS if Blank and diluent doesn't interfere | Pass |
| | with the main peaks. | |
| | PASSif coating agent used to perform the | |
| | APSD analysis doesn't interfere with the | |
| | main peaks. | |
| | PASS if the main peak of the POPG is | |
| | separated from the main impurities peak | |
| Resolution | — | 8.31 for peak RT 7.577 |
| | | 7.91 for peak RT 2.652 |
| Plates USP | — | 3616.42 |
| Tailing factor | <2 | 2 |
| Asymmetry 10% | — | 2.11 |

### APSD Method Development

The Next Generation Impactor (NGI) with USP throat was used to develop the aerodynamic particle size distribution APSD for the DPI device performed as stipulated for DPI products in the USP <601>. The Next Generation Pharmaceutical Impactor (NGI) is a modern impactor well suited for efficient laboratory use and even automated analysis. The NGI has been designed to have sharp cut-off curves for efficient particle size discrimination.

In order to avoid reentrainment, the collection surface of the NGI plates need to be coated, which is a pharmacopopeial requirement for DPIs. Particles should have efficient impactor stage capture avoiding reentrainment to give good accuracy and low variability when determining the correct particle size distribution. The stage capture must also be efficient over the entire flow rate range tested.

Three variations of collections cup surface were considered for the test, including and uncoated surface:
1% Silicon oil in Hexane;
1% Silicon oil in Heptane;
1% Tween 20 in Ethanol.

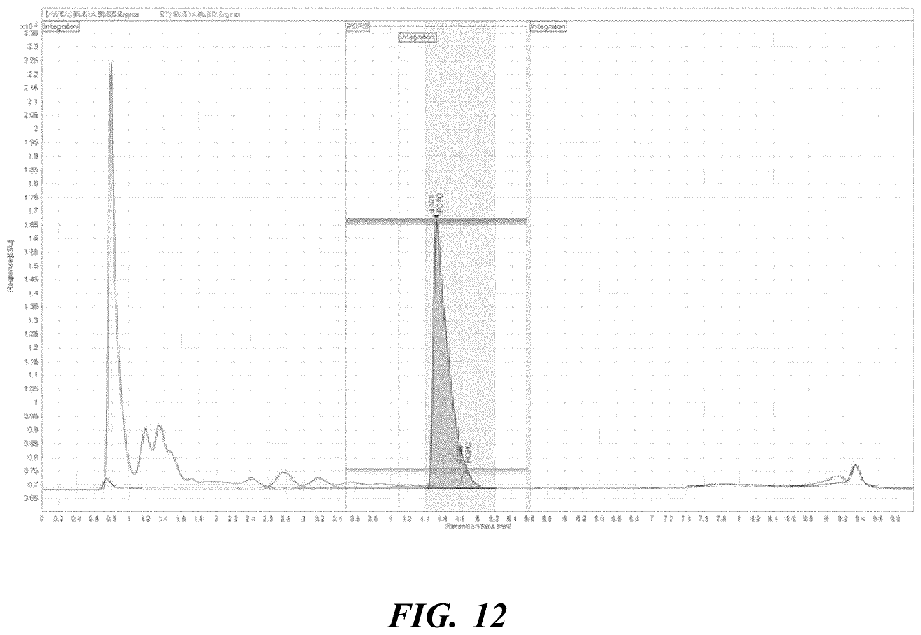

Due to the hydrophobic nature of these coating agents, the silicon oil was seen to be interfering with the POPG peak, interfering with the specificity requirement for the chromatography. The Tween 20 (1%) in Ethanol did not seem to interfere with the main API (FIG. 12).

Due to the interference of the silicon oil with the POPG, APSDs were performed using as a coating agent Tween 20 in Ethanol and without coating agent.

The spray dried powder 174 #005A-01 was filed into a size 2 vegetarian capsules with a mass of 80 mg. The APSD performance was done using an RS01 high resistance device size 2.

The flow rate was set up to 64 L/min with an actuation time of 3.8 seconds.

The procedure for the API recovery from the NGI was performed as per Table 9

TABLE 9
Volume used for the APSD recovery

| Stage | Diluent Volume/mL |
|---|---|
| Capsules | 50 |
| Device | 100 |
| Mouthpiece | 20 |
| Induction port | 100 |
| S1 | 20 |
| S2 | 20 |
| S3* | 50 |
| S4* | 50 |
| S5* | 50 |
| S6 | 10 |
| S7 | 10 |
| S8 (MOC) | 10 |

*A two stages recovery was done for the stages 3-5. The initial 20 mL were transferred in a 50 mL volumetric flasks. A further 10 mL were added to the NGI plates and recovered in the same 50 mL volumetric flask. The additional dilution were performed to have the area results within the calibration curve assessed.

Based on this preliminary results is suggested the usage of Tween 20 in Ethanol as coating agent in order to avoid reentrainment of the powder and reduce variability.

### HPLC DDU Method Development

A Delivered Dose Uniformity (DDU) method was developed for the DPI device performed as stipulated for DPI products in the USP <601>. The DDU method development was carried out using a DUSA equipment, a 47 mm metal filter was placed at the end of the DUSA tube and the flow rate was adjusted as required for the testing to give an inhaled volume of 2 L.

The spray dried powder 174 #005A-01 was filed into a size 2 vegetarian capsules with a mass of 80 mg and actuated in the DUSA tube. The recovery of the POPG from the DUSA was effected following the procedure below:
Remove the DUSA put the cap to seal the filter.
·Remove the mouthpiece adapter and wash with 10 mL of IPA into the DUSA.
Insert 40 mL of IPA into the DUSA.
Seal the DUSA with the other lid and shake it well manually for 2 minutes.
Transfer the content from the DUSA in a 500 mL amber volumetric flasks.
Add 50 mL of IPA in the DUSA tube and repeat step 4 to 6.
Repeat step 6 further 4 times.
Add 100 mL of Chloroform to the same 500 mL amber volumetric flasks.
Sonicate for 10 minutes
Allow to cool down and make up to volume using chloroform.
Dilute 5 mL of the sample in a 10 mL amber volumetric flak and make up to volume with diluent

### Formulation Screening & Feasability Assessments

### Solubility Screening for API Spray Drying

The solubility of the surfactant was investigated to identify a suitable solvent system in which the surfactants can be solubilized to process the material by spray drying.

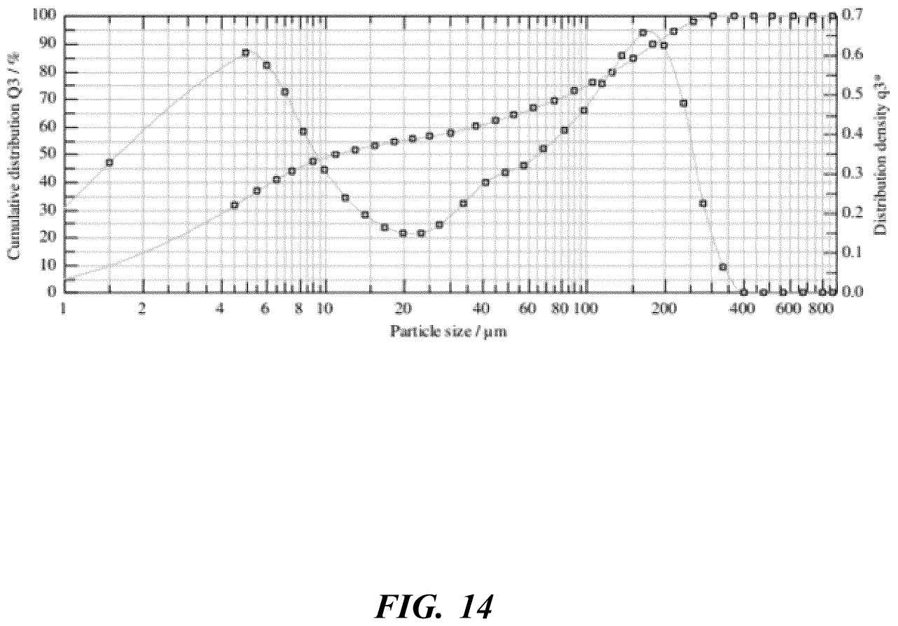

The solubility test is summarised in TABLE 10 and FIG. 14. The class III solvents were preferred to the class II solvents because the class II solvents may be regarded as less toxic and of lower risk to human health. The solubility of the POPG using class III solvent was lower than 0.5 mg/mL. For this reason, Class II solvents were investigated. The highest solubility was obtained with a mixture DCM/Methanol 5:1 v/v and it will be used as a vehicle for the spray drying process.

TABLE 10
POPG solubility results.

| | | | Boiling | | |
|---|---|---|---|---|---|
| | | Limit | point | Solubility | |
| Solvent | Class | (pp) | (° C.) | (mg/mL) | Comment |
| Ethanol (96%) | III | — | 78 | 0.5 | Initially appeared insoluble at 0.5 mg/mL. Fully |
| | | | | | dissolved when observed next day when observed |
| | | | | | next day. |
| Ethyl Acetate | III | — | 77 | <0.5 | Insoluble at 0.5 mg/mL. Undissolved aggregates. |
| | | | | | Formed fine, cloudy suspension when sonicated. |
| Acetone | III | — | 56 | <0.5 | Insoluble at 0.5 mg/mL. Undissolved aggregates. |
| | | | | | Formed fine, cloudy suspension when sonicated. |
| Isopropanol (IPA) | III | — | 82.5 | <0.5 | Insoluble at 0.5 mg/mL. Undissolved aggregates. |
| | | | | | Formed fine, cloudy suspension when sonicated. |
| Dimethyl sulfoxide | III | — | 189 | 0.5 | Dissolved following sonication. |
| (DMSO) | | | | | |
| Heptane | III | — | 98 | <0.5 | Insoluble at 0.5 mg/mL. Formed fine, cloudy |
| | | | | | suspension. |
| Methanol | II | 3000 | 65 | 1.35 | Clear Solution. |
| Dichloromethane | II | 600 | 40 | <0.5 | Insoluble at 0.5 mg/mL. Formed fine, cloudy |
| (DCM) | | | | | suspension. |
| Dimethylformamide | II | 880 | 153 | <0.5 | Insoluble at 0.5 mg/mL. Mostly dissolved |
| (DMF) | | | | | following sonication. |
| Cycloexhane | II | 3880 | 81 | <0.5 | Insoluble at 0.5 mg/mL. Formed fine, cloudy |
| | | | | | suspension. |
| Chloroform | II | 60 | 61 | 16.7 | Dissolved following extended vortex mixing. |
| DCM/ Methanol 5:1 | II | — | — | ~70 | Dissolved |
| v/v | | | | | |

### Spray Drying Feasibility Studies

The incoming API material was characterized initially using the following physicochemical properties techniques:
Particle size distribution (PSD) analysis—to determine the quality of the source API in terms of particle size
Modulated Differential Scanning calorimetry (mDSC)—to determine the thermotropic properties of the API to help select suitable spray drying inlet and outlet temperatures.
X-ray powder diffraction (XRPD)
Scanning electron micrographs (SEM)

### Particle Size Distribution

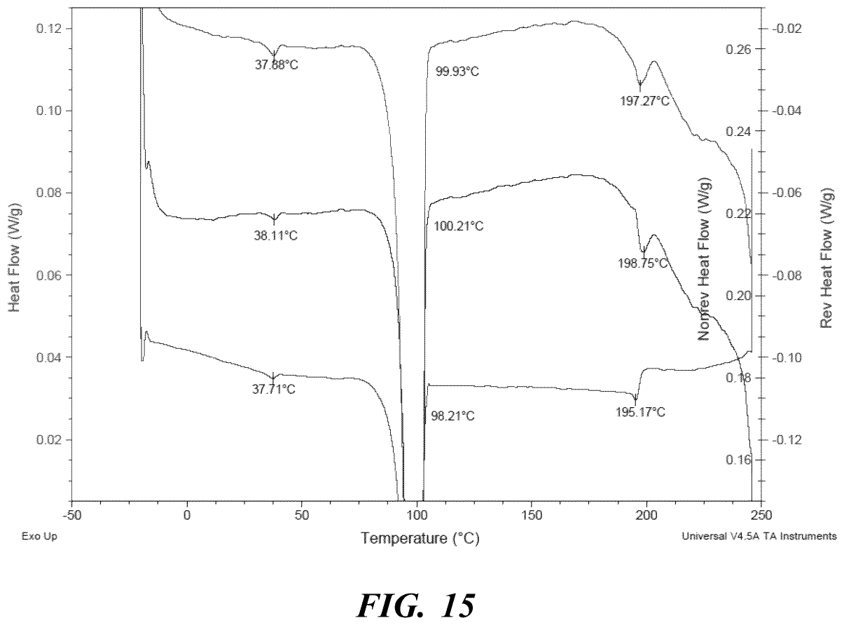

The Particle Size Distribution analysis was performed at 3 bar dispersion pressure using a Sympatec laser Particle Sizer equipped with RODOS/ASPIROS dispersion unit and an R5 lens. The results are summarised in TABLE 11 and in FIG. 15. The API had a broad, bimodal distribution.

TABLE 11
PSD results of the POPG raw material

| Sample | X10 (μm) | X50 (μm) | X90 (μm) | VMD( μm) |
|---|---|---|---|---|
| POPG | 1.80 ± 0.07 | 12.65 ± 4.65 | 183.21 = 33.83 | 59.05 ± 13.17 |

### Differential Scanning Calorimetry

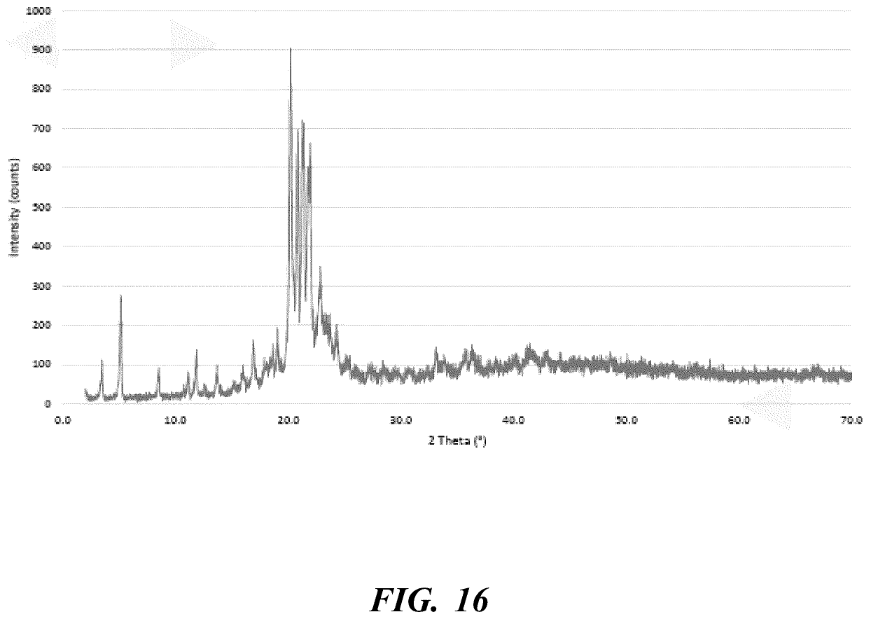

DSC analysis was performed using a TA Instruments Q20 MDSC with auto sampler and refrigerated cooling accessory. The DSC result is summarised in FIG. 16.

A small endothermic event at ˜38° C. was observed and this could be indicative of molecular relaxation. Reversible heat flow suggested possible presence of Tg. An endothermic event as observed ˜100° C. and is indicative of melting transition.

### X-Ray Powder Diffraction (XRPD)

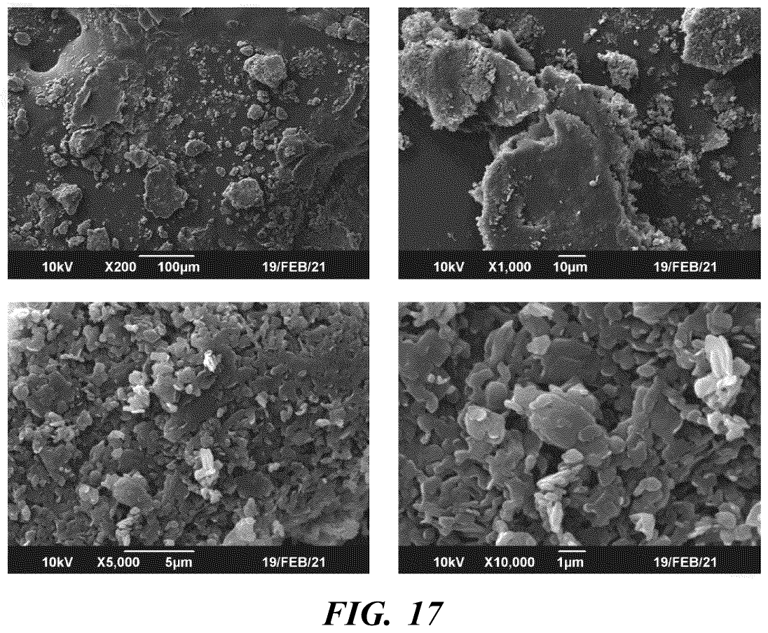

The X-ray powder diffraction (XRPD) pattern of the POPG samples, was employed to investigate check the crystallinity. The XRPD result is summarised in FIG. 17. The result suggest that the API contain a mix of amorphous and crystalline material. The sharp peaks are due to the crystalline component and the broad features (sometimes referred to as “halo”) to the amorphous component.

### Scanning Electron Micrographs (SEM)

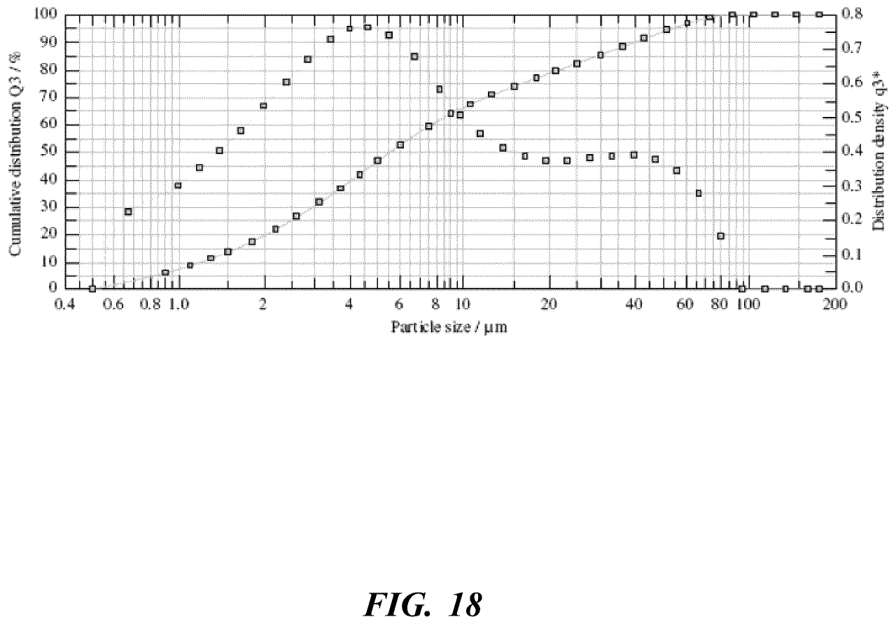

The SEM analysis of the POPG raw material, showed in FIG. 18, shows the presence of aggregates of wide ranges of sizes and each particle exhibits a complex irregular shape.

### Spray Drying Process

All feasibility batches were produced on a 5 g scale, using a Buchi B-290 laboratory spray dryer with a standard Buchi two-fluid nozzle (0.7 mm nozzle tip). The drying temperature was be optimised based on initial DSC studies performed on the incoming material and based on the first spray dried batches.

The spray dried batches were prepared from a feed solution at 5% w/v solids in 5:1 (v/v) DCM:MeOH. The Solution spray dried using Buchi B290 spray dryer according to TABLE 12.

TABLE 12
Spray drying process and results.

| | Inlet | Outlet | Atomisation | Feed rate | Yield |
|---|---|---|---|---|---|
| Batch | (° C.) | (° C.) | pressure (bar) | (g/min) | (%) |
| 174#003A1 | 91-92 | 60 | 4 | 5 | 57.2 |
| 174#003B1 | 74-75 | 50 | 5 | 5 | 46.6 |
| 174#005A1 | 54-55 | 40 | 5 | 5 | 60.3 |

The spray dried batch 174 #003A-01, was collected in good yield as a white powder with good flow properties. Losses were due to deposition of material in cyclone (collected −6.1%) and spray chamber and exhaust filter bag (fines).

The spray dried batch 174 #003B-01, was collected in good yield as a white powder with good flow properties. The product yield was lower than the first batch, possibly due to increased fines lost exhaust filter at higher atomisation pressure. Some losses due to deposition of the material in the cyclone accounted for around 3.7% of the yield.

The spray dried product 174#005A1, was collected in good yield as a white powder with good flow properties. The product yield, at 60.3%, was higher than the than the first two batches manufactured previously.

### Particle Size Distribution of the Spray Dried Product

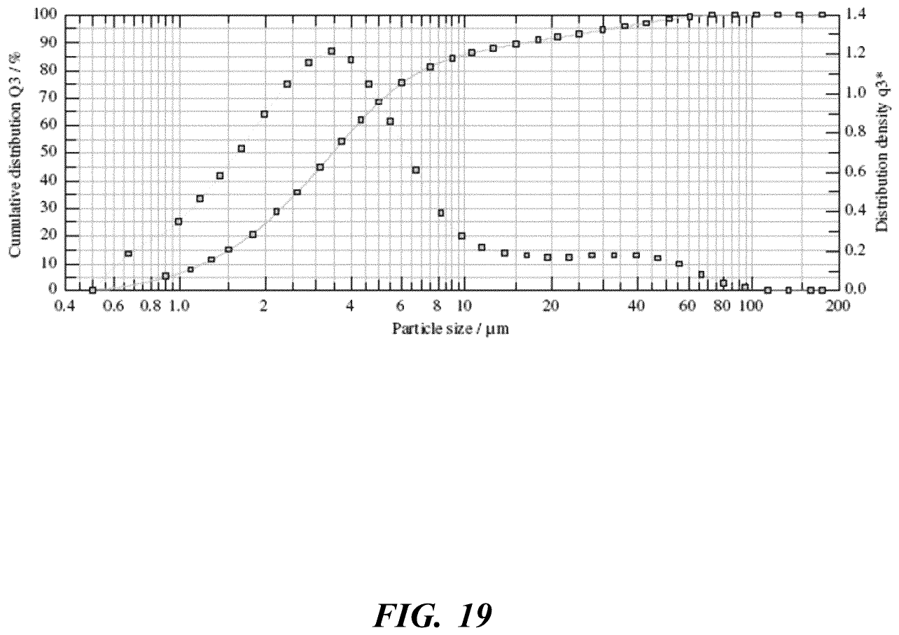

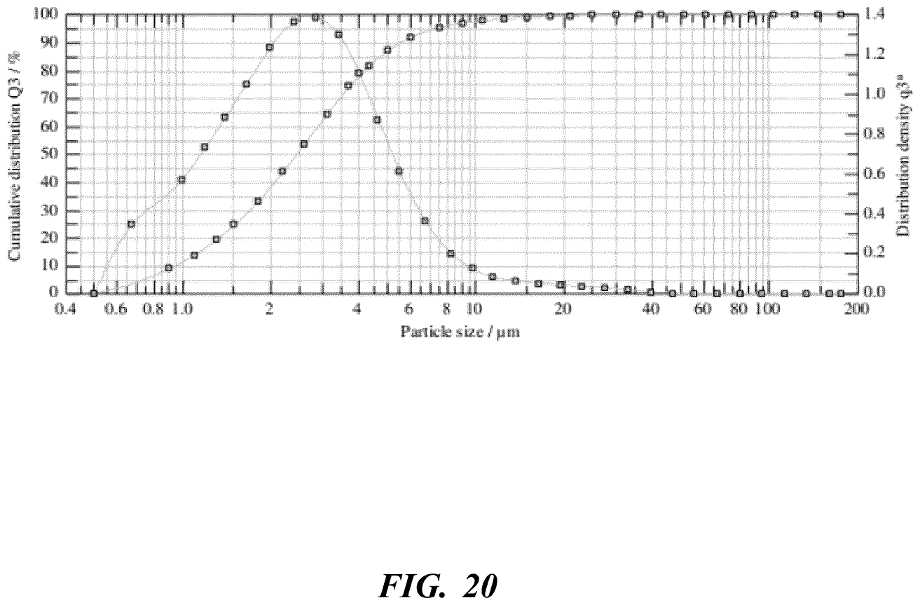

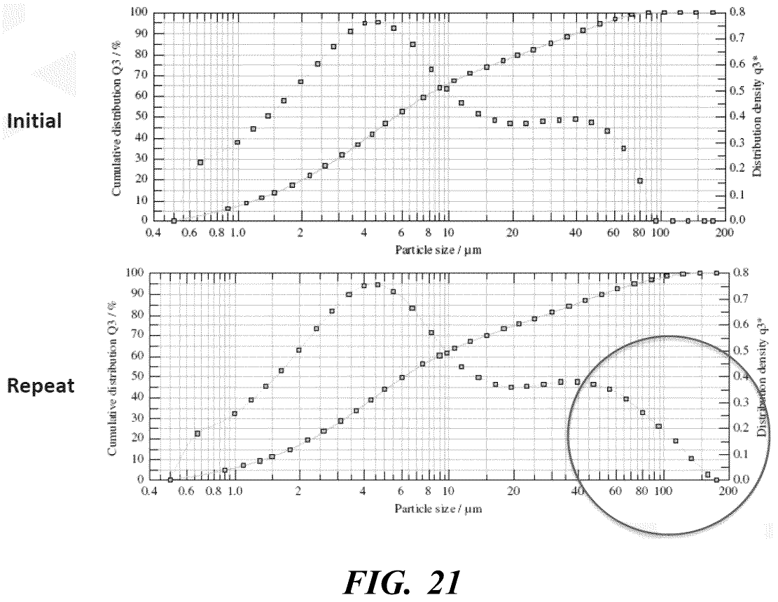

The particle size analysis of the spray dried powders was performed at 3 bar dispersion pressure using a Sympatec laser Particle Sizer equipped with RODOS/ASPIROS dispersion unit and an R3 lens (n=2) and is summarised in Table 13 and in FIG. 19-FIG. 21. The PSD of spray dried powders decreased with increasing atomisation pressure and reducing outlet temperature during the spray drying manufacture.

TABLE 13
PSD of the spray dried product of the the batch 174#003A-01

| Sample | X10 (μm) | X50 (μm) | X90 (μm) | VMD( μm) |
|---|---|---|---|---|
| 174#003A1 | 1.23 | 5.57 | 40.24 | 13.34 |
| 174#003B1 | 1.24 | 3.46 | 17.32 | 7.26 |
| 174#005A1 | 0.95 | 2.46 | 5.64 | 3.17 |

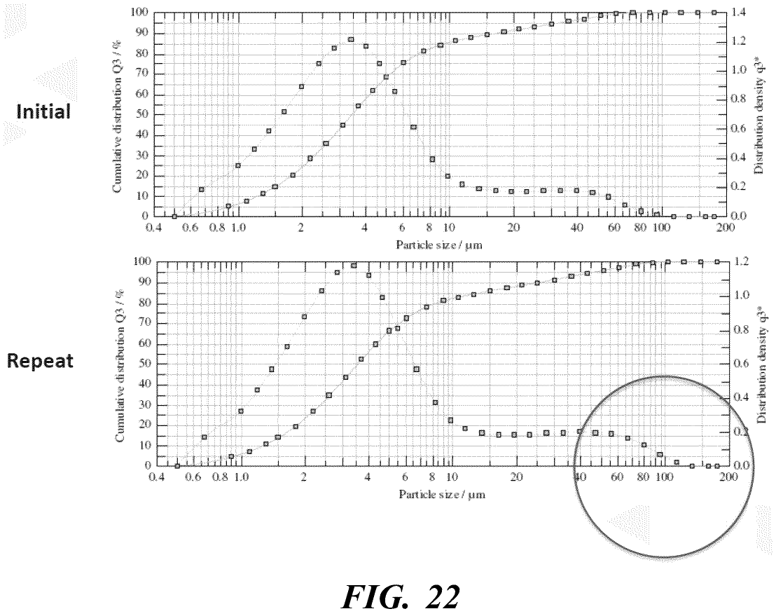

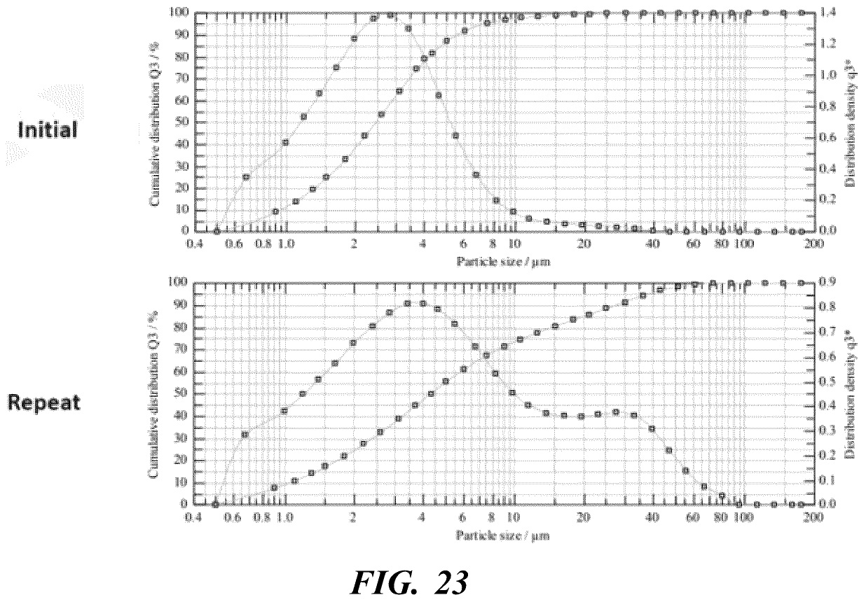

The PSD analysis performed on 174 #003A-01 & 174 #003B-01 following storage at −20° C. was repeat-ed. The results are summarised in FIGS. 22-23 and in TABLE 14.

TABLE 14
PSD of the spray dried products for the batches 174#003A-01 and
174#003B-01.

| Sample | | X10 (μm) | X50 (μm) | X90 (μm) | VMD(μm) |
|---|---|---|---|---|---|
| 174#003A-01 | Initial | 1.23 | 5.57 | 40.24 | 13.34 |
| 174#003A-01 | Repetition | 1.39 | 6.19 | 52.98 | 17.70 |
| 174#003B-01 | Initial | 1.24 | 3.46 | 17.32 | 7.26 |
| 174#003B-01 | Repetition | 1.28 | 3.56 | 25.97 | 9.26 |

The Dv90 of both spray dried formulations appeared to have increased; likely due to increase in aggregated material.

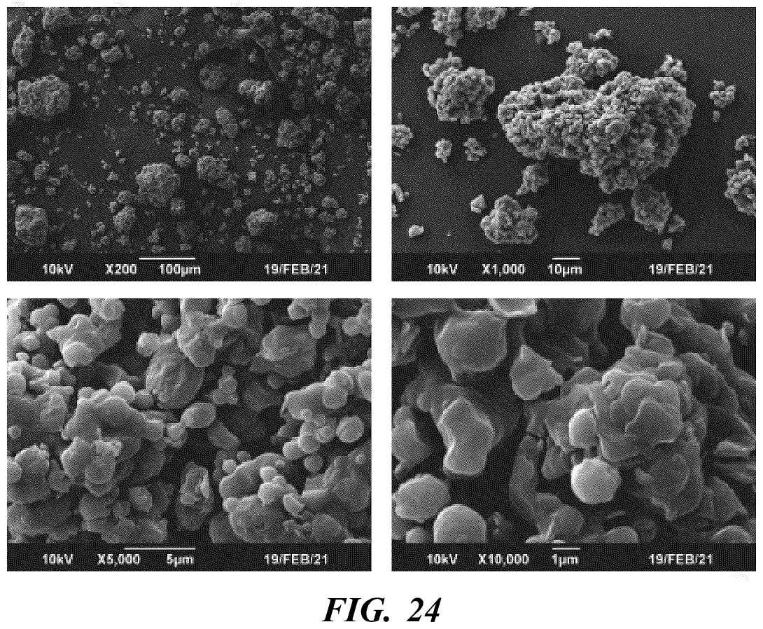

The spray dried product 174 #005A-01 was vacuum dried for 24 hours at 25° C. with a maximum pressure ˜0.1 mbar. After the 24 hours, the PSD of this product was evaluated. Post vacuum drying, increased amounts of aggregated material were observed and as a result the Dv90 increased. The results are summarised in TABLE 15 and in FIG. 24.

TABLE 15
PSD of the spray dried products for the batch 174#005A-01.

| | | X10 | X50 | X90 | VMD |
|---|---|---|---|---|---|
| Sample | | (μm) | (μm) | (μm) | (μm) |
| 174#005A-01 | Initial | 0.95 | 2.46 | 5.64 | 3.17 |
| 174#005A-01 | Repetition post | 1.07 | 4.33 | 28.43 | 9.63 |
| | vacuum drying | | | | |

Based on the PSD results after the storage, an aliquot of the formulation 174 #005A-01, that was stored at −20° C. for 4 weeks, was taken out from the freezer, transferred in sealed container and equilibrated for 5 hours at ambient temperature (20 C-25 C, <30% RH) in a sealed vial. The PSD of this sample was measured and the results are summarised in Table 16. After the equilibration the powder showed disaggregation with the Dv90 even smaller than the one post spray drying. Based on this data it was concluded that the storage condition and the equilibration time played an important factor on the PSD.

TABLE 16
PSD of the spray dried products for the batch 174#005A-01.

| | | X10 | X50 | X90 | VMD |
|---|---|---|---|---|---|
| Sample | | (μm) | (μm) | (μm) | (μm) |
| 174#005A-01 | After 5 hours | 0.85 | 2.19 | 4.77 | 2.79 |
| | equilibration at | | | | |
| | ambient temperature | | | | |

### Gas Chromatography (GC) Analysis

The GC analysis was performed on the vacuum dried samples to determine residual MeOH/DCM content within each formulation. The results is summarised in Table 17. The LOD was 0.5 ppm and the LOQ was 2 ppm

TABLE 17
GC of the spray dried products.

| | DCM (PPM) | MeOH (PPM) |
|---|---|---|
| Batch | (Specification = <600 PPM) | (Specification = <3000 PPM) |
| 174#003A1 | No Detection (<LOD) | No Detection (<LOD) |
| 174#003B1 | No Detection (<LOD) | No Detection (<LOD) |
| 174#005A1 | No Detection (<LOD) | No Detection (<LOD) |

### Assay by UPLC-ELSD for the Spray Dried Product

The Assay method was assessed with UPLC coupled with and ELSD detector and used as a fast quantitative method. For the test 10 mg of each of the spray dried products were dissolved in a 200 mL volumetric flasks. The results are summarised in TABLE 18.

TABLE 18
Assay results of the spray dried products.

| | Average amount of POPG | Mass balance |
|---|---|---|
| Batch | recovered (μg) | (% LC) |
| 174#003A1 | 10257.93 | 102.58 |
| 174#003B1 | 10162.75 | 101.63 |
| 174#005A1 | 10125.98 | 101.26 |

### Scanning Electron Microscope (SEM) Spray Dried Product

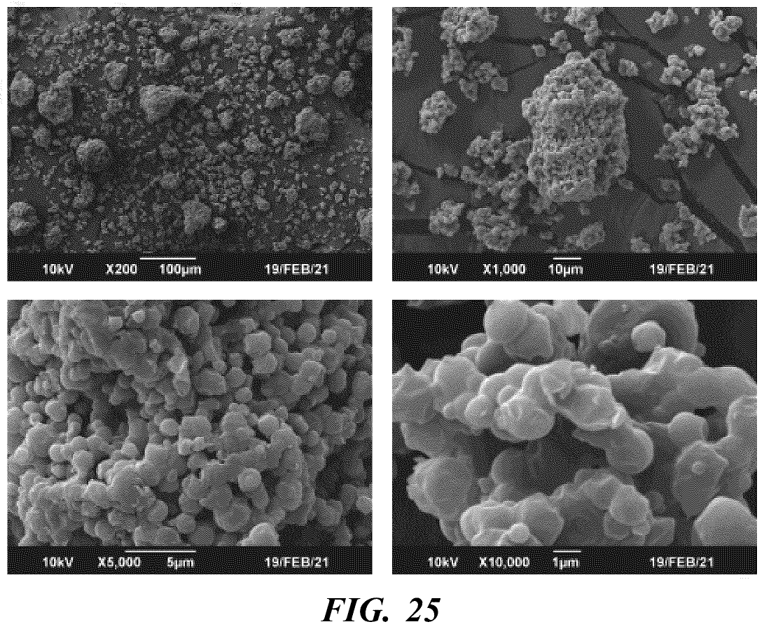

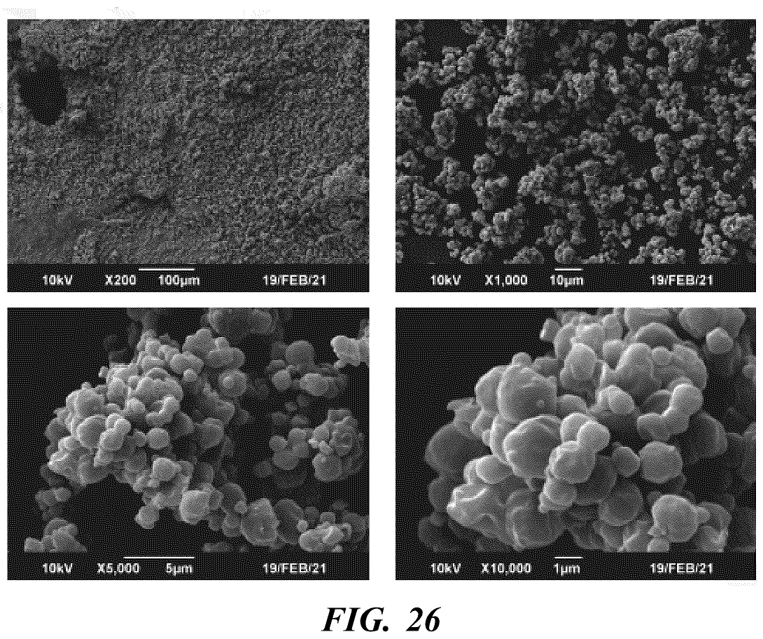

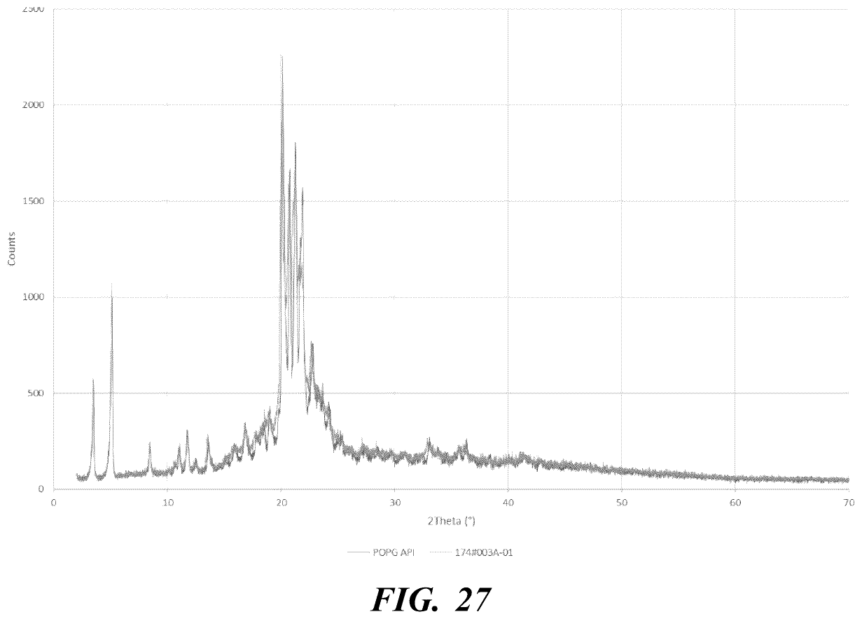

SEM was performed on the spray dried product (see FIGS. 25-27). Batch 174 #003A1 exhibits a complex irregular shape with large aggregates where the particles are bonded. Batch 174 #003B1 exhibits a complex irregular shape with large aggregates where the particles are bonded. Batch 174 #005A1 exhibits a complex smaller aggregates compared with the two previous SD powder.

### Xray Powder Diffraction (XRPD) Spray Dried Product

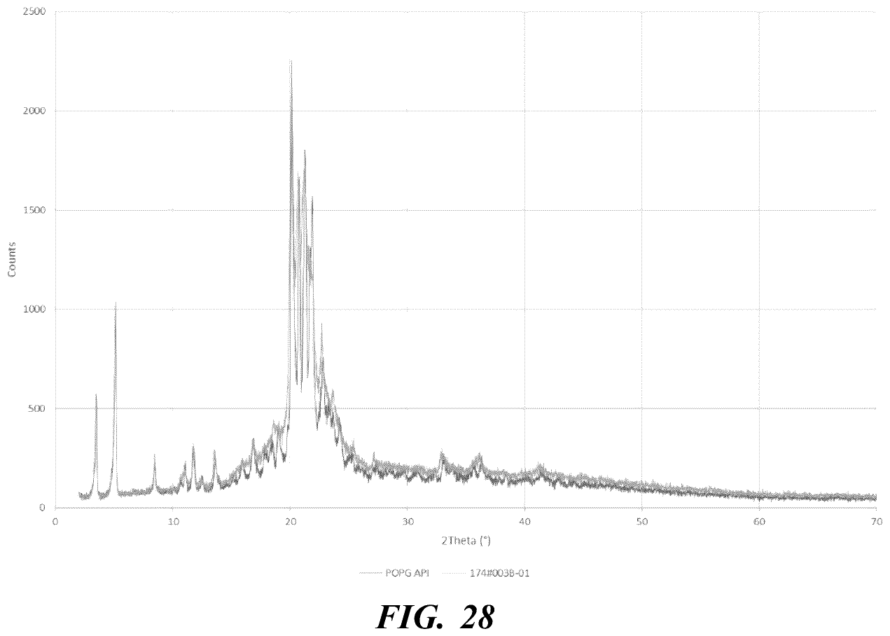

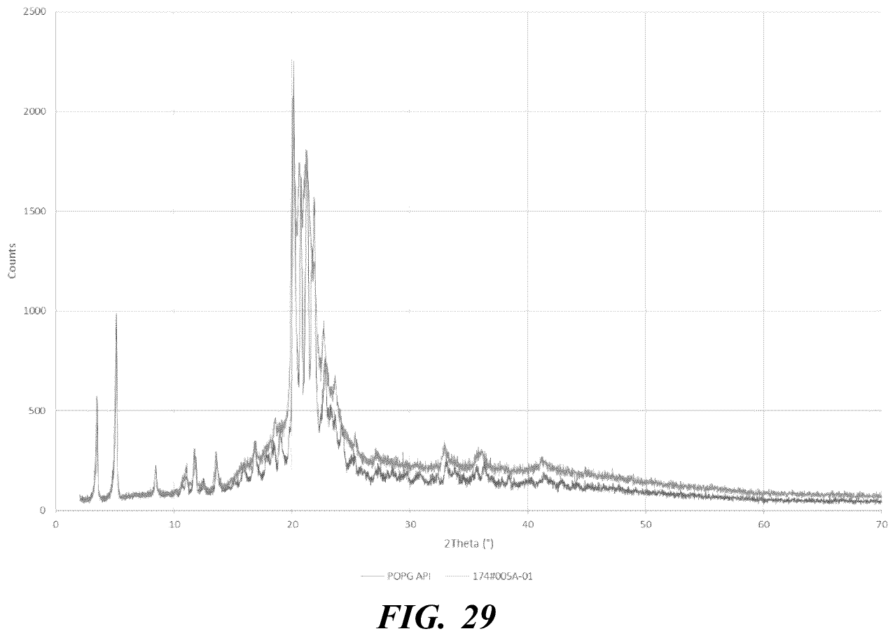

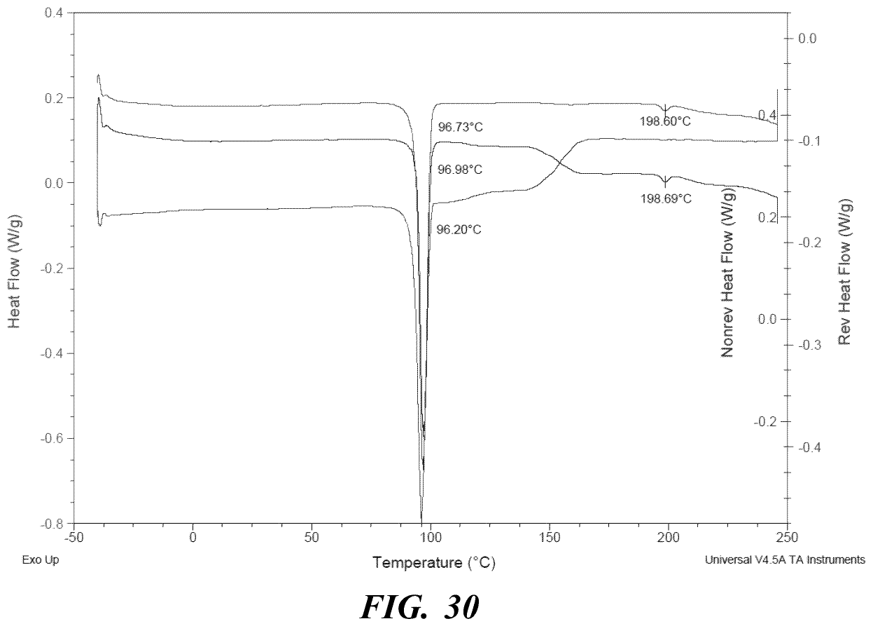

The XRPD of the POPG raw material and the corresponding spray dried sample is summarised in FIGS. 28-30. The results showed that no difference was observed in the degree of crystallinity comparing the XRPD of the raw API and the corresponding spray dried material.

### Water Content Determination by Karl Fischer (KF) Titrators

The water content analysis was carried out placing in the Karl Fischer system, coupled with an oven, 50 mg of the spray dried powders. The results of the water content analysis is summarised in Table 19. The SD powder is highly hygroscopic with low water content, around 0.4%.

TABLE 19
Water content results of the Spray dried products.

| Batch | Water content (%) |
|---|---|
| 174#003A1 | 0.37 ± 0.03 |
| 174#003B1 | 0.41 ± 0.05 |
| 174#005A1 | 0.37 ± 0.07 |

### Spray Drying Manufacture Process Robustness

The robustness of the spray dried manufacture process was verified manufacturing an additional batch on a 5 g scale, using a Buchi B-290 laboratory spray dryer with a standard Buchi two-fluid nozzle (0.7 mm nozzle tip) with the purpose to obtain a spray dried product with similar PSD to the 174 #005A1. The spray dried batches were prepared from a feed solution at 5% w/v solids in 5:1 (v/v) DCM:MeOH. The solution spray dried using Buchi B290 spray dryer according to TABLE 20.

The spray dried product 174 #010A-01 was vacuum dried for 24 hours at 25° C. After the 24 hours, the PSD of this product was evaluated.

TABLE 20
Spray drying process of the batch 174#003A-01

| | Inlet | Outlet | Atomisation | Feed rate | Yield |
|---|---|---|---|---|---|
| Batch | (° C.) | (° C.) | pressure (bar) | (g/min) | (%) |
| 174#010A-01 | 54-55 | 40 | 5 | 5 | 52 |

The PSD of the batch was measure pre and post vacuum drying and the results are summarised in TABLE 21.

TABLE 21
PSD of 174#010A-01 post spray drying and post vacuum drying

| | | X10 | X50 | X90 | |
|---|---|---|---|---|---|
| Sample ID | | (μm) | (μm) | (μm) | VMD(μm) |
| 174#010A-01 | Post spray | 0.74 | 1.79 | 3.64 | 2.04 |
| | drying | | | | |
| | Post vacuum | 0.74 | 1.79 | 3.67 | 2.12 |
| | drying | | | | |

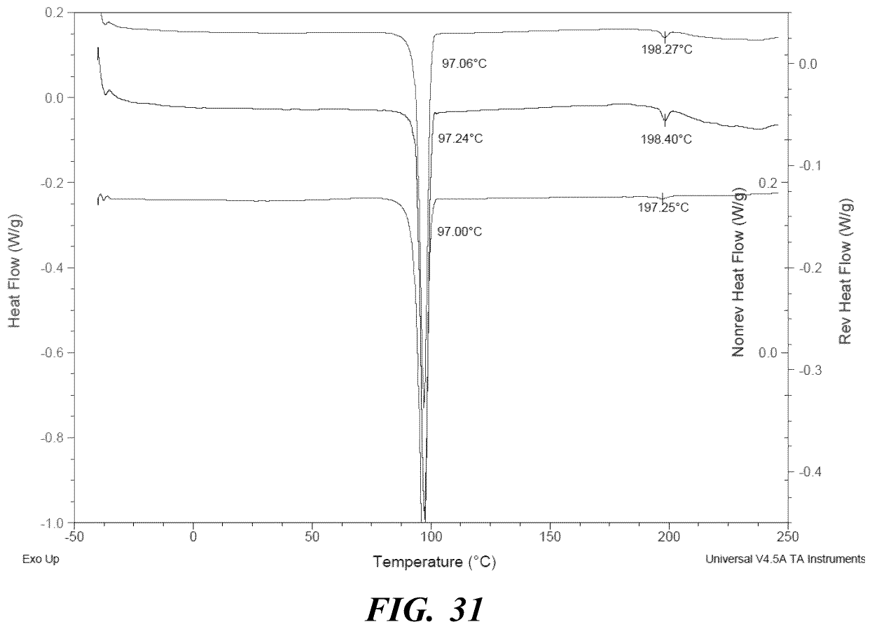

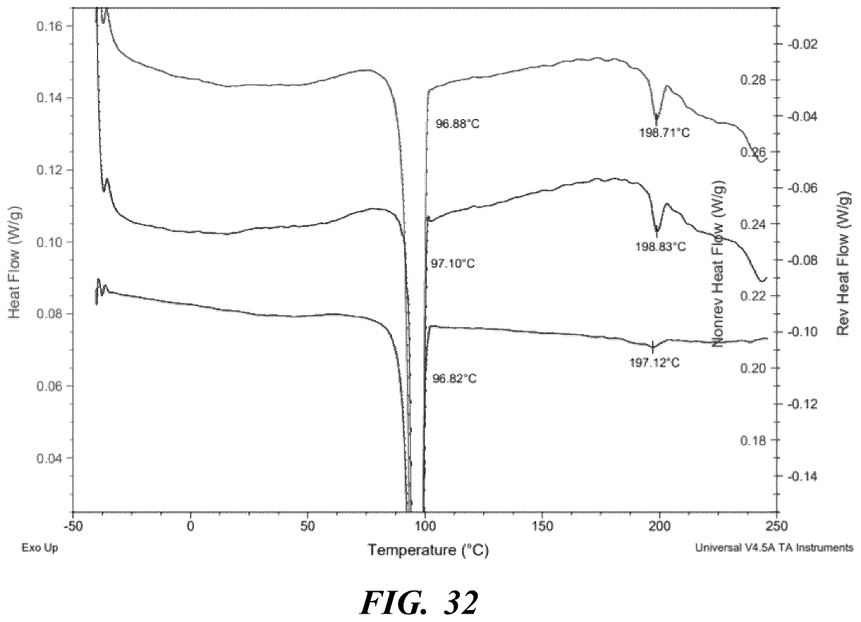

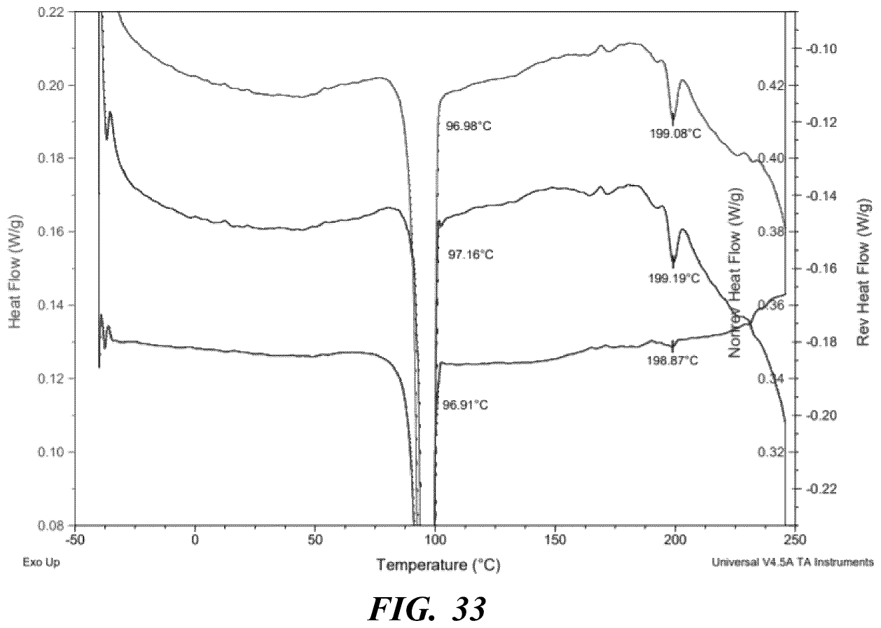

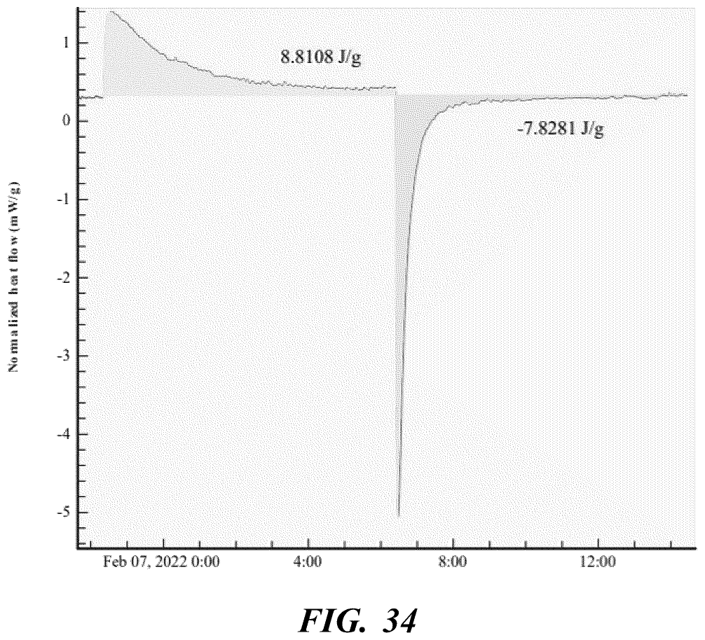

The 174 #010A-01 post vacuum drying was separated in different aliquot and stored at 2-8° C. and at −20° C. The PSD and DSC of the spray dried powder was evaluated through time. The spray dried powder was removed from the storage condition an equilibrated at low RH for 5 hours (19° C. and 28% RH) before to perform any test. The PSD and the DSC are summarised in TABLEe 22 and in FIGS. 31-34. The PSD results suggests that the PSD after T=14 days (post re-equilibration) was similar to the PSD after the manufacture. The DSC results suggest the presence of and endothermic event as observed around 100° C., indicative of melting transition. The reverse heat flow DSC post spray drying showed a change in the baseline that anyway was identified as an artefact rather than a physical change in the sample. After t=12 days the DSC results didn't suggest any crystalline change when compared with the DSC post spray drying-post vacuum drying.

TABLE 22
PSD of 174#010A-01 post storage, evaluation through time.

| | Timepoint | | | X10 | X50 | X90 | VMD |
|---|---|---|---|---|---|---|---|
| Sample ID | (days) | Condition | | (μm) | (μm) | (μm) | (μm) |
| 174#010A-01 | T = 1 | 2-8° | C. | 0.74 | 1.81 | 3.79 | 2.15 |
| | T = 1 | −20° | C. | 0.74 | 1.81 | 3.74 | 2.26 |
| | T = 7 | 2-8° | C. | 0.74 | 1.81 | 3.68 | 2.06 |
| | T = 7 | −20° | C. | 0.76 | 1.87 | 3.89 | 2.29 |
| | T = 14 | 2-8° | C. | 0.76 | 1.85 | 3.70 | 2.09 |
| | T = 14 | −20° | C. | 0.75 | 1.84 | 3.79 | 2.17 |

The spray dried powder was evaluated performing an assay analysis and the results are summarised in Table 23. No difference was observed after 14 days testing.

TABLE 23
Assay results of 174#010A-01.

| Material ID | Timepoint (days) | Condition | Assay (%) |
|---|---|---|---|
| 174#010A-01 | 0 | −20° C. | 98 ± 0.45 |
| | 14 | 2-8° C. | 93 ± 0.23 |

### Bulk Density Measurement

The bulk density of batch 174 #010A-01 was assessed following the storage stability study using spare samples stored at 2-8° C. and −20° C. which were equilibrated at room temperature (20% RH) for >5 hours and then pooled into a single aliquot for analysis. Initial attempts to measure the bulk density of 174 #010A-01 using a 10 mL measuring cylinder proved unsuccessful as the highly statically charged powder coated the inside of the glassware, making it impossible to perform measurements without additional sample agitation. As a result of this, the material was re-collected and stored overnight 2-8° C. in an attempt to dissipate the static charge. Following overnight storage at 2-8° C., the pooled material was re-equilibrated at room temperature (20% RH) for >5 hours in the presence of an anti-static gun. Unfortunately, the powder maintained a high static charge, which made bulk density measurement impossible using the standard method. A small-scale bulk density assessment was attempted using a 1.5 mL Eppendorf. The material was observed to adhere to the walls of the Eppendorf and form a cavity in the middle; again, making any meaningful measurements impossible (FIG. 35).

In order to estimate the achievable fill weight of the spray dried material in size 3 HPMC capsules, the pooled sample of 174 #010A-01 was hand-filled in to size 3 capsules using a micro-spatula. Approximately 67-68 mg of material was filled into the capsule without tamping; with light tamping, a fill weight of 84 mg was achieved. However, adhesion of material to the walls of the capsule was observed, and a slight cavity formed in the center (FIG. 36). Considering that the volume of the HPMC size 3 capsule is around 0.27 mL the bulk density after a light tamping is 0.322 g/ml.

### Stage 3B: Micronisation of POPG

A single 7 g aliquot of POPG (unformulated) was initially de-aggregated using a 1 mm sieve and then passed through an Attritor M3 fluid energy mill at high venturi/grinding pressures, with the aim of achieving the target particle size distribution of X50 2-3 μm. The micronisation parameters are summarized in TABLE 24.

TABLE 24
Micronisation process.

| | Venturi | Grind | Feed rate | Yield |
|---|---|---|---|---|
| Sample ID | Pressure (bar) | Pressure (bar) | (g/min) | (%) |
| 174#013A-01 | 8.0 | 2-2.5 | 2.42 | 87.4 |

The particle size distribution of the micronized sample was determined by laser particle size analysis using an R1 (0.1-35.0 μm range) & R3 lens (0.5-175.0 μm range) at 3 bar dispersal pressure at T-0 and after T=7 days after storage of the sample at −20° C. Before analyzing the sample at −20° C., the micronized product was allowed to equilibrate at room temperature for >5 hours. The formulation generally appeared to be within target size range. There was a slight discrepancy between the two lenses, with the trace of the material measured using the R1 lens appearing larger and very slightly bimodal which wasn't apparent in the trace measured using an R3 lens. This may have been due to the increased sensitivity of the R1 lens vs the R3 at this particle size.

The additional usage of the R1 lens was due to the smaller nature of the micronized batch. For this reason the 174 #013A-01 was analysed using and R3 and R1 lens. The R1 lens permit higher resolution at this particle size. The PSD results are summarized in TABLE 25.

TABLE 25
PSD results of 174#013A-01.

| | Timepoint | | X10 | X50 | X90 | VMD |
|---|---|---|---|---|---|---|
| Sample ID | (days) | Lens | (um) | (um) | (um) | (um) |
| 174#013A-01 | T = 0 | R1 | 0.67 | 2.07 | 5.75 | 2.70 |
| | T = 7 d −20° C. | R1 | 0.67 | 2.44 | 5.97 | 2.95 |
| | T = 14 d −20° C. | R1 | 0.56 | 1.48 | 3.28 | 1.83 |
| | T = 0 | R3 | 0.61 | 1.12 | 2.35 | 1.34 |
| | T = 7 d −20° C. | R3 | 0.63 | 1.26 | 2.87 | 1.55 |
| | T = 14 d −20° C. | R3 | 0.64 | 1.34 | 3.07 | 1.65 |

Based on the PSD results, further measurements would be required to establish whether the increases are due to particle aggregation or just natural variation of the material.

Whilst measurements performed using the R3 lens remained fairly consistent across all timepoints, there was a notable decrease in particle size observed for the T=14 day storage sample sized using the R1 lens vs T=0 & T=14 days. The result was more comparable to those obtained using the R3 lens.

The micronized powder was evaluated performing an assay analysis and the results are summarised in TABLE 26.

TABLE 26
Assay results of 174#013A-01.

| Material ID | Timepoint (days) | Condition | Assay (%) |
|---|---|---|---|
| 174#013A-01 | 7 | −20° C. | 94 ± 2.91 |

### Bulk Density Measurement of 174 #013A-01

The bulk density of batch 174 #013A-01 was assessed following the storage stability study using spare samples stored at −20° C. In order to estimate the achievable fill weight of the micronized material in size 2 HPMC capsule, the pooled sample of 174 #013A-01 was hand-filled into size 2 capsules using a spatula. Approximately 111 mg of material was filled into the capsule with tamping; however, adhesion of material to the walls of the capsule was observed, (FIG. 37). Considering that the volume of the HPMC size 2 capsule is around 0.36 mL the bulk density after a light tamping is 0.308 g/ml.

TAM analysis of spray dried POPG powder batch 174 #010A-01 was performed to determine the amorphous content. The TAM measures the heat changes associated with a process or an event e.g. recrystallisation at a constant temperature. Perfusion isothermal microcalorimetry allows the investigation of recrystallisation of API powders using vapour adsorption, whilst measuring the heat change over time as the material undergoes re-crystallisation. The vapour can be water and is controlled using a series of mass flow controllers that introduce a defined vapour pressure of the solvent into a hermetically sealed vial containing the powder. A thermal activity monitor was used to analyse the POPG sample. Amorphous content analysis was carried out using TA instruments TAM II with a 4 mL Gas Perfusion Cell. The test method was used with the following cycling: 1%-90%-1% over a 6 hour-8 hour-8 hour period with a sample size of 50±1 mg. The POPG samples presented the following water absorption and desorption obtaining a wetting peak ˜8.81 J/g and a drying peak a ˜−7.82 J/g. Overall the POPG sample seem to have a mixture of crystalline in structure with a low amorphous content exhibited by the differences between the wetting and drying peak with a value of ˜0.98 J/g, that represent the amorphous content. To be able to identify the percentage % of amorphous content, a reference standard of the material that is 100% crystalline and 100% amorphous is required which is not readily available. For this reason, the spray dried material can be evaluated in terms of phase change but the relative amorphous content cannot be quantified.

TABLE 27
Spray Drying Parameters

| | | | Wetting |
|---|---|---|---|
| | | | peak- |
| | Wetting Peak | Drying Peak | Drying peak |
| Sample | Integral (J/g) | Integral (J/g) | (J/g) |
| 174#010A- | 8.8108 | −7.8281 | 0.9827 |
| 01 | | | |

The stability of the spray drying feed solution at 5% w/v POPG in 5:1 (v/v) DCM:MeOH was assessed. The feed solution was evaluated at day 0,1,2,3,7 at two different storage conditions:
25° C./60% RH
2° C.-8° C.

The results obtained are shown in TABLE 28 below:

TABLE 28
Spray Drying Results

| | | Time- | Mass | Total |
|---|---|---|---|---|
| | Storage | point | balance | impurities |
| Formulation | conditions | (days) | (%)** | (%) |
| Feed solution | 25° C./60% RH | 0 | 98.75 | 0.00 |
| at 5% | 2° C.-8° C. | | | |
| w/v POPG | 25° C./60% RH | 1 | 98.63 | 0.00 |
| in 5:1 (v/v) | 2° C.-8° C. | | 93.85* | 0.00 |
| DCM:MeOH | 25° C./60% RH | 2 | 98.31 | 0.00 |
| | 2° C.-8° C. | | 100.06 | 0.00 |
| | 25° C./60% RH | 3 | 99.16 | 0.00 |
| | 2° C.-8° C. | | 99.34 | 0.00 |
| | 25° C./60% RH | 7 | 96.89 | 0.00 |
| | 2° C.-8° C. | | 97.56 | 0.00 |

*Lower value obtained to analytical error. Values on consequent days show no degradation.
**Mass balance and impurity profiles were obtained by HPLC ELSD quantification methods.

After doing the analysis at different conditions during 7 days, the feed solution at 5% w/v POPG in 5:1 (v/v) DCM:MeOH showed stability at both conditions studied, 25° C./60% RH and 2° C.-8° C. up to three days. This is shown by an absence of impurities detected, as well as a mass balance (%) recovery ranging between 98.31% and 100.06%. After this period, even if the presence of impurities was not detected, the mass balance (%) dropped to values of 96.89 and 97.56 for 25° C./60% RH and 2° C.-8° C. conditions, respectively. However, this could be explained as well because of certain inconsistencies regarding the HPLC assay method, not necessarily meaning that impurities were present.

### CONCLUSIONS

The chemical-informatics analysis showed that the solubility for POPG was at a minimum level between approximately pH 0.0 and pH 12.0. The analysis showed that the aqueous solubility of POPG increases significantly in alkaline solutions where the pH is 14.0 it reached the maximum solubility (in excess of ˜580 mg·mL).

An Assay method for POPG was successfully developed to quantify the emitted dose. The calibration curve in the range between 1.14-146.29 μg/mL, as a free base, assessed follows a cubic regression model.

A matrix of co-solvents was performed and the results suggested that the combination DCM/Methanol 5:1 v/v was used to obtain a solubility of ˜ 70 mg/mL. Based on this results three spray dried batches were manufactured from a feed solution at 5% w/v solids in 5:1 (v/v) DCM:MeOH.

The POPG raw material was characterized through DSC, SEM, and PSD. Based on the POPG characterization three spray dried formulations were manufactured adjusting the process parameters to obtain a PSD between 2-5 μm. The residual solvent for DCM and MEOH was lower than the LOQ.

The batch 174 #005A-01 manufacture showed the best properties in terms of PSD, with a Dv50 of 2.46 μm. The Spray drying process as well didn't seem to have an impact on the degree of crystallinity of the material. The SEM analysis showed that this powder had smaller aggregates compared with the other two batches manufacture and it presented spherical particles of different size. It was observed that the storage conditions and the re-equilibration time of the powder are key factors that impacts the PSD.

Based on this previous observation a new batch was manufactured, 174 #010A-01, using the same spray drying parameters used for the product 174 #005A-01. The PSD was evaluated through time and after 14 days storage and re-equilibration it was observed no variation on the PSD results when compared to the time zero analysis. The DSC analysis didn't show any transitions phase comparing the powder spray drying and after 14 days storage.

A single 7 gr batch of POPG was successfully micronized and the PSD evaluated.

An assay evaluation was performed on 174 #010A-01 and 174 #013A-01 and no difference was observed. Despite there was no difference on the Assay data, the 174 #013A-01 showed different results on the PSD when evaluate through time. The physical stability of this product should be evaluated.

The bulk density of the 174 #010A-01 and 174 #013A-01 showed as well similar bulk density based on the capsule filling obtained.

TAM analysis shows low amorphous content.

Spray drying feed solution at 5% (w/v) of POPG in DCM: Methanol 5:1 was found to be stable for 3 days at 25 C/60% RH and 2-8 C without any degradation.

Various phospholipids may be utilized in the compositions and methodologies disclosed herein. Preferably, the phospholipid is an anionic lipid having (1) a hydrophobic portion; (2) a negatively charged portion; and (3) an uncharged, polar portion. Specific, nonlimiting examples of anionic lipids useful in the compositions and methodologies disclosed herein include, but are not limited to, unsaturated phosphatidylglycerol, unsaturated phosphatidylinositol, saturated short chain phosphatidylglycerol, saturated short chain phosphatidylinositol, and derivatives of any of such phospholipids (e.g., polyethylene glycol (PEG) conjugates of these phospholipids), as well as anionic sphingolipids, anionic glycerolipids (anionic diglycerides, such as SQV-diglyceride), and derivatives of such lipids. Preferred phospholipids include, but are not limited to, unsaturated phosphatidylglycerol, unsaturated phosphatidylinositol, palmitoyl-oleoyl-phosphatidylglycerol (POPG), and dimyristoyl-phosphatidylglycerol (DMPG). In one preferred embodiment, the phospholipids are selected from palmitoyl-oleoyl-phosphatidylglycerol (POPG) and/or phosphatidylinositol (PI) and/or derivatives thereof.

Another class of phospholipids which may be utilized in the compositions and methodologies disclosed herein feature xylitol-headgroup phospholipids (also referred to herein as “xylitol lipid analogs”, and “xylitol-headgroup lipid analogs”), specifically xylitol-headgroup analogs of palmitoyl-oleoyl-phosphatidylglycerol (POPG).

The xylitol lipid analogs disclosed herein preferably have (a) a phospholipid glycerol backbone, (b) a xylitol polar headgroup, a phosphodiester bond linking the glycerol backbone to the xylitol polar headgroup, and (c) variable hydrophobic regions comprising two aliphatic chains of 14 to 18 carbons in length, wherein linkage between the aliphatic chains and the phospholipid glycerol backbone is an O-acyl linkage or an O-alkyl linkage, and further wherein the aliphatic chains contain 0 to 2 double bonds. The addition of the xylitol to diacylglycerol-phosphate, or the dialkylglycerol-phosphate, creates a chiral center at the 3 position of the xylitol, thus the resulting compounds are more specifically 3′R and 3'S mixtures of the headgroup xylitol. These xylitol lipid analogs include dimyristoyl-phosphatidylxylitol (DMPX; also referred to as: sn-1-myristoyl, sn-2-myristoyl sn-3 glycerol phosphoxylitol); 14:0 Diether-phosphatidylxylitol (14:0 DEPX; also referred to as: sn-1-O-myristyl, sn-2-O-myristyl sn-3 glycerol phosphoxylitol); Dipalmitoyl-phosphatidylxylitol (DPPX; also referred to as: sn-1-palmitoyl, sn-2-palmitoyl sn-3 glycerol phosphoxylitol); palmitoyl-oleoyl-phosphatidylxylitol (POPX; also referred to as: sn-1-palmitoyl, sn-2-oleoyly sn-3 glycerol phosphoxylitol); 16:1 Dipalmitoleoyl-phosphatidylxylitol (16:1 DPPX; also referred to as: sn-1-palmitoleoyl, sn-2-palmitoleoyl sn-3 glycerol phosphoxylitol); distearoyl-phosphatidylxylitol (DSPX; also referred to as: sn-1-stearoyl, sn-2-stearoyl sn-3 glycerol phosphoxylitol); dioleoyl-phosphatidylxylitol (DOPX; also referred to as: sn-1-oleoyl, sn-2-oleoyl sn-3 glycerol phosphoxylitol); 18:1 Diether-phosphatidylxylitol (18:1 DEPX; also referred to as: sn-1-O-octadecenyl, sn-2-O-octadecenyl sn-3 glycerol phosphoxylitol); and dilinoleoyl-phosphatidylxylitol (DLPX; also referred to as: sn-1-linoleoyl, sn-2-linoleoyl sn-3 glycerol phosphoxylitol; also referred to herein as 18:2 DLPX).

As described herein, compositions containing an “effective amount” of an anionic lipid or related compound of the invention contain an amount of the specific anionic lipid or related compound effective to inhibit an inflammatory process in vitro or in vivo, or to inhibit viral infection in vitro or in vivo, as measured by any suitable technique for measuring such activity, several of which are known to the art. See, for example, U.S. Pat. No. 10,532,066 (Voelker et al.) and U.S. Pat. No. 11,278,556 (Voelker), which are incorporated herein by reference in their entirety.

In some embodiments, the compositions and methodologies disclosed herein feature a composition comprising a (preferably homogeneous) lipid preparation of the anionic lipid(s) or related compound(s) disclosed herein and at least one additional agent. The additional agent may include any pharmaceutical carrier or an additional agent for the treatment of inflammation or pathogen infection (e.g., an anti-viral agent).

Suitable anti-inflammatory agents include, but are not limited to, cytokine inhibitors, chemokine inhibitors, chemoattractant inhibitors, Cox inhibitors, leukotiene receptor antagonists, leukotriene synthesis inhibitors, inhibitors of the p38 MAP kinase pathway, glucocorticoids. More specifically, anti-inflammatory compounds can include, but are not limited to: any inhibitor of eicosanoid synthesis and release, including any Cox-2 inhibitor; Cox-1 inhibitors; inhibitors of some certain prostaglandins (prostaglandin E(2); PGD(2)), inhibitors of certain leukotrienes (LTB4); classes of antibiotics with known direct or indirect anti-inflammatory effects, including macrolides (e.g. azithromycin) and fluoroquinolones (e.g., levofloxacin; moxifloxacin; gatifloxacin); inhibitors of p38 MAP kinase; inhibitors of the function of pro-inflammatory cytokines and chemokines, including antagonists of tumor necrosis factor (TNF), antagonists of interleukin-8 (IL-8); transforming growth factor beta (TGF-beta), β-agonists (long or short acting), antihistamines, phosphodiesterase inhibitors, corticosteroids, and other agents.

As used herein, the term “pharmaceutically acceptable carrier” includes pharmaceutically acceptable excipients and/or pharmaceutically acceptable delivery vehicles, which are suitable for use in the administration of a preparation, formulation or composition, including a liposomal composition or preparation, to a suitable in vivo site. A suitable in vivo site is preferably any site wherein inflammation or infection by a pathogen, for example, is occurring or is expected to occur. Preferred pharmaceutically acceptable carriers are capable of maintaining a formulation of the invention in a form that, upon arrival of the formulation at the target site in a patient (e.g., the lung tissue), the formulation is capable of acting at the site, preferably resulting in a beneficial or therapeutic benefit to the patient. A delivery vehicle for a protein or agent may include the lipid preparation itself, if another agent is included, although in most embodiments of the invention, the lipid preparation is also a therapeutic agent as described herein (e.g., the lipid preparation can serve one or both functions).

Suitable excipients for use in the compositions and methodologies disclosed herein include excipients or formularies that transport or help transport, but do not specifically target, a composition or formulation to a cell or tissue (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Formulations of the present invention can be sterilized by conventional methods and/or lyophilized.

The compositions and methodologies disclosed herein may be utilized to treat, prevent or inhibit inflammation or infections associated with particular toll-like receptors such as, for example, TLR1, TLR2, TLR3, TLR4, TLR6, TLR7, TLR8, and/or TLR10. These TLRs have been associated, for example, with various bacterial infections, protozoan and fungal infections, viral infections e.g., Cytomegalovirus infection, Herpes simplex virus infection, measles, Varicella-zoster virus infection, HIV infection, rhinovirus infection, parainfluenza virus infection, Human parechovirus infection, influenza type A viral infection, Papilloma virus infection), cancer (including, but not limited to, melanoma), and autoimmune diseases. Accordingly, the compositions and methodologies disclosed herein may be utilized to treat or inhibit inflammation associated with any of these conditions or to prevent or inhibit infection by a pathogen associated with any of these conditions.

The above description of the present invention is illustrative and is not intended to be limiting. It will thus be appreciated that various additions, substitutions and modifications may be made to the above described embodiments without departing from the scope of the present invention. Accordingly, the scope of the present invention should be construed in reference to the appended claims. For convenience, some features of the claimed invention may be set forth separately in specific dependent or independent claims. However, it is to be understood that these features may be combined in various combinations and subcombinations without departing from the scope of the present disclosure. By way of example and not of limitation, the limitations of two or more dependent claims may be combined with each other without departing from the scope of the present disclosure.

What is claimed is:

A1. A method for treating an infection or inflammation in a subject, comprising:
administering to the subject, via an oral inhalation route, an amount of a composition that is effective to inhibit said infection or to treat said inflammation, wherein the composition contains a phospholipid.

A2. The method of claim A1, wherein said phospholipid is phosphatidylglycerol.

A3. The method of claim A4, wherein said phosphatidylglycerol is POPG.

A4. The method of claim A1, wherein the composition is prepared by a spray drying process.

A5. The method of claim A1, wherein the composition has a PSD between 2-5 μm.

A6. The method of claim A1, wherein the infection is an RSV, Influenza or HRV infection.

A7. The method of claim A1, wherein the composition can be used to treat inflammation, allergic asthma, COPD, ALI, Sepsis.

A8. The method of claim A1, wherein the composition is effective to inhibit infection in the subject.

A9. The method of claim A1, wherein the composition is effective to inhibit inflammation in the subject.

A10. The method of claim A1, wherein the composition is effective to inhibit both infection and inflammation in the subject.
